SEQUENCE LISTING (5 sequences)

SEQ ID NO: 1 - DNA, 20 bp, Artificial sequence
  Synthetic Oligonucleotide Sequence
acactgggggggctagggaa

SEQ ID NO: 2 - DNA, 20 bp, Artificial sequence
  Synthetic oligonucleotide sequence
caaaacgtattttgtacaat

SEQ ID NO: 3 - DNA, 60 bp, Artificial sequence
  Synthetic oligonucleotide sequence
ttccctagcccccccagtgtgcaagggcagtgaagacttgattgtacaaaatacgttttg

SEQ ID NO: 4 - DNA, 21 bp, Artificial sequence
  Synthetic oligonucleotide sequence
actgctagacattttccacat

SEQ ID NO: 5 - DNA, 21 bp, Artificial sequence
  Synthetic oligonucleotide sequence
actgctagagattttccacat

(19) United States
(12) Patent Application Publication
(10) Pub. No.: US 2008/0292545 A1
(43) Pub. Date: Nov. 27, 2008

(54) Functionalized Encoded Apoferritin Nanoparticles and Processes for Making and Using Same

(76) Inventors: Yuehe Lin, Richland, WA (US); Guodong Liu, Fargo, ND (US); Hong Wu, Richland, WA (US); Jun Wang, Richland, WA (US); Darrell R. Fisher, Richland, WA (US)

Correspondence Address: BATTELLE MEMORIAL INSTITUTE;ATTN: IP SERVICES, K1-53, P. O. BOX 999, RICHLAND, WA, 99352, US

(21) Appl. No.: 12/062,745

(22) Filed: Apr. 4, 2008

Related U.S. Application Data
(60) Provisional application No. 60/910,056, filed on Apr. 4, 2007.

(51) Int. Cl. A61K 9/14; A61K 39/395; A61K 39/00; A61K 38/00; A61K 31/7088; A61K 51/08; A61K 49/04; A61K 49/14; A61K 49/00; G01N 33/53; C12Q 1/68

(52) U.S. Cl. 424/1.29; 424/490; 424/130.1; 424/184.1; 514/12; 424/9.34; 424/9.6; 436/501; 436/518; 514/44

(57) ABSTRACT

Apoferritin nanoparticles with functionalized surfaces have been prepared that include preselected agents within the cavity of the apoferritin molecule and preselected functionalized surface characteristics on the outer surface of the nanoparticle. Such materials provide for utilization and selective modification in a variety of applications including therapeutic and diagnostic uses. Examples of several of these applications are described herein. In addition a method for the creation of these materials by alternatively assembling, functionalizing, or functionalizing, disassembling and reassemblying the materials provides for creative customization of various types of materials applicable for varying types of applications which are also described herein.

### CROSS REFERENCE TO RELATED APPLICATION

This application claims priority from Provisional application No. 60/910,056 filed 4 Apr. 2007, incorporated in its entirety herein.

This invention was made with Government support under Contract DE-AC0676RLO-1830 awarded by the U.S. Department of Energy. The Government has certain rights in the invention.

### FIELD OF THE INVENTION

The present invention relates generally to apoferritin nanoparticles and more particularly to encoded apoferritin nanoparticles with functionalized surfaces, and methods for making and using same. The invention finds application in, e.g., protein and DNA biosensors; and carriers for imaging and treating disease states, e.g., cancer.

### BACKGROUND OF THE INVENTION

The enormous quantity of information generated in the Human Genome and Proteomic Project has generated tremendous demands for innovative analytical tools capable of delivering genetic and proteomic information at the sample source. The present invention provides a material that enables various applications in meeting these needs.

### SUMMARY OF THE INVENTION

In one aspect, the invention includes a functionalized apoferritin nanoparticle that surrounds various preselected agents within the apoferritin nanoparticle that encode the nanoparticle with preselected properties and functionality. “Preselected agent” as used herein means any component or constituent that when introduced to the core (cavity) of the apoferritin nanoparticle provides a desired effect or function, whether chemical, physical, and/or biological; or endows the apoferritin nanoparticle with preselected properties and functionality as described further herein. Preselected agents include, but are not limited to, e.g., metals and metal-containing constituents. Metal containing constituents include metals selected from the Group IA metals; Group IIA metals; Group I-B metals; Group II-B metals; Group III-B metals; Group IV-B metals; Group V-B metals; Group VI-B metals; Group VII-B metals; Group III-A metals; and combinations of these metals; metal salts (e.g., metal phosphates); diagnostic and radiotherapeutic agents (e.g., lutetium-177, yttrium-90, and other like radioisotopes); therapeutic agents (e.g., drugs or other pharmaceuticals); agents; oncology agents; imaging agents; contrast agents (e.g., fluorescence markers such as fluorescein-containing salts); redox agents (e.g., redox markers such as hexacyanoferrate-containing salts); electroactive agents (e.g., hexacyanoferrate-II and hexacyanoferrate-III ions, Cd²⁺, Pb²⁺, Bi²⁺ and other metal cations, or electroactive agents); electrochemical agents; calorimetric agents, (e.g., dyes); optically-active agents; magnetic agents (e.g., magnetic particles); paramagnetic agents; and the like, including combinations of listed agents. The surface of the apoferritin nanoparticle can be functionalized with various molecules and chemical constituents including, but not limited to, e.g., proteins (e.g., avidin, streptavidin, etc.); peptides; haptens; aptamers; nucleic acids (e.g., DNA), nucleotides; esters (e.g., N-hydroxy-succinimide ester); antibodies (e.g., anti-TNF-α antibody); antigens; vitamins and cofactors (e.g., biotin); and various combinations of listed constituents. Conjugates that attach to apoferritin nanoparticles include, e.g., proteins (e.g., avidin, streptavidin, etc.); peptides; haptens; nucleic acids (e.g., DNA), nucleotides; aptamers; esters (e.g., N-hydroxy-succinimide ester); antibodies (e.g., anti-TNF-α antibody); antigens; vitamins and cofactors (e.g., biotin); and various combinations of listed constituents, e.g., antibody-hapten-peptide conjugates. Functionalization of the surface of the nanoparticle is achieved with various coupling reagents including, e.g., 3-(3-dimethylaminopropyl)-1-ethylcarbodiimide (EDC) and biotinamidohexanoyl-6-amino-hexanoic acid N-hydroxy-succinimide (NHS) ester (i.e., Biotin-NHS), as well as other methodologies and reagents as will be known to those of skill in the art.

The present invention also includes processes for making functionalized apoferritin nanoparticles that are encoded with preselected agents. These processes include the steps of: surrounding a preselected agent with an apoferritin molecule that defines an apoferritin nanoparticle. Surface of the apoferritin nanoparticle is functionalized with preselected constituents as described herein. In one process, preselected agents are introduced into, and surrounded by, the core of the nanoparticle by disassembling the apoferritin nanoparticle into subunits and reassembling to encapsulate (encode) the preselected agents. Preselected agents can also be introduced to the apoferrtin cavity (core) by diffusion. Various combinations of preselected agents can also be introduced to an apoferritin nanoparticle by a combination of encapsulation and diffusion processes. Preselected agents can also be released from the core of the apoferritin nanoparticle. These functionalities provide for a variety of features and capabilities. For example, in one embodiment the release of one or more metal cations from the core of an encoded apoferritin nanoparticle generates an electrochemical signal that can be measured under suitable conditions in an electrochemical process or device. This feature may be incorporated with other features in applications such as assays for the detection of materials such as proteins, nucleic acids, and other detection sensitive molecules; in immunoassay processes and devices (e.g., for quantification of single-nucleotide polymorphisms; and antibody-antigen recognition events); probe devices for detection of nucleic acids; biochip array processes and devices (e.g., for detecting DNA, proteins, and other biomolecules); radioimmunodetection processes, and devices; radioimmunotherapy processes and devices; electrochemical processes and devices; voltammetric processes and devices; product identification and authenticity processes and devices; product tracking processes and devices; imaging processes and devices, therapeutic agents, pharmaceutical agents and drugs, and radioisotopes, e.g., for detection and treatment of tumors and cancers; and combinations of listed applications, processes, and devices.

While the present invention is described herein with reference to preferred embodiments thereof, it should be understood that the invention is not limited thereto, and various alternatives in form and detail may be made therein without departing from the scope of the invention. A more complete appreciation of the invention will be readily obtained by reference to the following description of the accompanying drawings in which like numerals in different figures represent the same structures or elements.

### BRIEF DESCRIPTION OF THE DRAWINGS

FIG. 1 illustrates an encapsulation process for making apoferritin nanoparticles encoded with metal phosphates as preselected agents, according to an embodiment of the invention.

FIG. 2 illustrates a diffusion process for making apoferritin nanoparticles encoded with metal phosphates as preselected agents, according to an embodiment of the invention.

FIGS. 3a-3c present electrochemical measurements of apoferritin nanoparticles encoded with single metal phosphates.

FIGS. 4a-4c present electrochemical measurements of apoferritin nanoparticles encoded with two metal phosphates.

FIG. 5 illustrates a process for functionalization of apoferritin nanoparticles encoded with preselected agents, according to an embodiment of the invention.

FIG. 6 illustrates a diffusion process for functionalization of apoferritin nanoparticles encoded with preselected agents, according to an embodiment of the invention.

FIG. 7 illustrates a process for preparing apoferritin nanoparticles encoded with fluorescence markers suitable for assays and immunoassays, according to an embodiment of the invention.

FIG. 8 shows electrochemical measurements of biotin functionalized apoferritin nanoparticles encoded with hexacyanoferrate incubated with an avidin-modified screen-printed electrode, suitable for assays and immunoassays.

FIG. 9 illustrates an electrochemical immunoassay protocol that employs biotin-functionalized apoferritin nanoparticles encoded with hexacyanoferrate, according to an embodiment of the invention.

FIG. 10 presents electrochemical measurements obtained from functionalized apoferritin nanoparticles encoded with an electrochemical agent for electrochemical immunoassay of target antigen, according to an embodiment of the invention.

FIG. 11 illustrates an electrochemical immunoassay protocol that employs biotin-functionalized apoferritin nanoparticles encoded with a metal phosphate, according to another embodiment of the invention.

FIGS. 12a-12f show electrochemical measurements from electrochemical immunoassays demonstrated with biotin-functionalized apoferritin nanoparticles encoded with a metal phosphate as a function of increasing concentration of a target (TNF-α) antigen, according to an embodiment of the invention.

FIGS. 13a-13c show electrochemical measurements from electrochemical immunoassays with target (anti-TNF-α and MCP-1) antibody-functionalized apoferritin nanoparticles encoded with preselected metal phosphates, suitable for use in electrochemical immunoassay, according to an embodiment of the invention.

FIG. 14 illustrates a protocol for radioimmunoassay, radioimmunoimaging, and radioimmunotherapy involving a functionalized apoferritin nanoparticle encoded with preselected radioisotopes, according to another embodiment of the invention.

FIG. 15 illustrates a process for preparing apoferritin nanoparticles functionalized with a DNA probe, suitable for electrochemical detection of DNA, according to an embodiment of the invention.

FIG. 16 illustrates a DNA hybridization protocol that employs DNA functionalized apoferritin nanoparticles of FIG. 15 and DNA functionalized magnetic particles for electrochemical detection of DNA single-nucleotide polymorphisms, according to another embodiment of the invention.

FIG. 17 presents results from electrochemical immunoassays that employ DNA functionalized apoferritin nanoparticles of FIG. 15 encoded with an electrochemical agent for detection of target DNA, according to another embodiment of the invention.

FIG. 18 illustrates a DNA hybridization protocol that employs nucleotide functionalized apoferritin nanoparticles for quantitative electrochemical assay of DNA single nucleotide polymorphisms, according to an embodiment of the invention.

FIG. 19 illustrates another DNA hybridization protocol that employs nucleotide functionalized apoferritin nanoparticles for quantitative electrochemical assay of DNA single nucleotide polymorphisms, according to another embodiment of the invention.

FIG. 20 presents data obtained from electrochemical detection of target DNA measured in conjunction with nucleotide functionalized apoferritin nanoparticles of FIG. 19.

FIGS. 21a-21c illustrate a biosensor of a simple design that employs functionalized apoferritin nanoparticles, according to an embodiment of the invention.

FIG. 22 illustrates a process that employs functionalized apoferritin nanoparticles encoded with a radioisotope as a radiotherapeutic agent for treatment of oncologic tumors, according to an embodiment of the invention.

### DETAILED DESCRIPTION OF THE INVENTION

The present invention is a functionalized apoferritin nanoparticle that can be assembled and customized for application in a variety of applications. In one application these functionalized apoferritin nanoparticles can be used as a template to prepare single and multiple component metal nanoparticles, each with distinct voltammetric signatures, and each applicable for various uses. The preparation of these materials through the encapsulation and diffusion processes described below enable the successful control of the multiple metal composition ratios in compositionally encoded nanoparticles and provide a useful addition to a variety of applications including particle-based product-tracking/identification/protection, multiplex electrochemical biosensors and bioassays, and various other applications. Detailed descriptions of these devices, their methods of creation and various exemplary uses are shown in the accompanying figures and described hereafter.

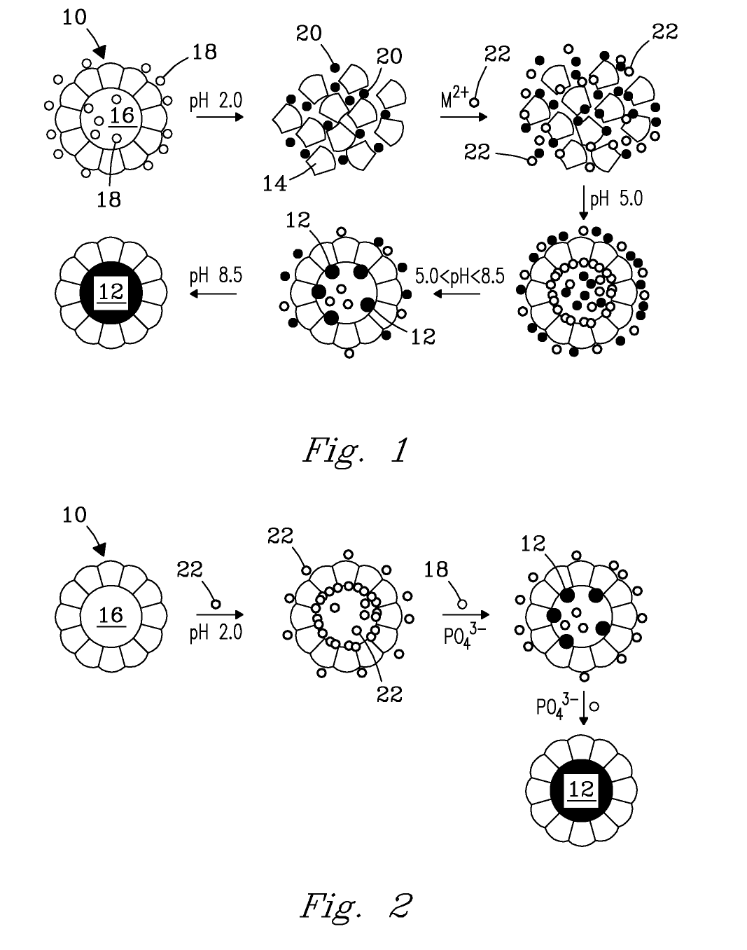

FIG. 1 illustrates an encapsulation process for encoding (loading) apoferritin nanoparticles 10 with preselected agents 12. Here, the process is described in reference to a metal phosphate as preselected agent 12, but is not limited thereto, as described further herein. In the figure, an apoferritin nanoparticle 10 is illustrated that is comprised of apoferritin subunits 14 that when assembled collectively define a cavity (core) 16. The apoferritin nanoparticle disassembles into subunits 14 in, e.g., a phosphate buffer saline (PBS) solution by adjusting pH to, e.g., pH 2. At this pH, phosphate ions (PO₄²⁻) 18 in the buffer react with hydrogen ions (not shown) to form dihydrogen phosphate anions 20. Metal ions 22 introduced into the buffer solution coexist with dihydrogen phosphate anions 20. At a pH of ˜5, apoferritin subunits 14 reassemble to form nanoparticle 10, which includes cavity 16. At this pH, metal cations 22 and dihydrogen phosphate anions 20 coexist in cavity (core) 16. Metal ions 22 concentrate along the inner surface of the apoferritin subunits 14 that define cavity 16. At a pH of between, e.g., 5.0<pH<8.5, phosphate ions 18 begin to precipitate with metal ions 22 forming seeds of metal phosphate 12 within the apoferritin cavity, which act as autocatalysts. Metal cations and phosphate anions outside the apoferritin cavity continue to diffuse into the cavity because of concentration differences. Thus, precipitation continues until cavity 16 fills with metal phosphate 12. Metal phosphate 12 encodes nanoparticle 10 with preselected functionality. Slow adjustment of pH provides for highly loaded apoferritin nanoparticles. Release of encoded metal ions 22 is effected with a change in solution pH, e.g., to a pH of 4.6. While the process has been shown here in reference to a single preselected agent, the process is not limited thereto. For example, reassembly of apoferritin subunits 14 in the presence of one or more preselected agents 12 surrounds a combination of agents within the apoferritin cavity 16, encoding the nanoparticle 10 with functionality provided by each of the preselected agents. The apoferritin nanoparticle remains stable during the encapsulation process. Encoded apoferritin nanoparticles are suitable for use in various sensors, devices, and/or assay applications, as described further herein.

A diffusion process for preparation of apoferritin nanoparticles encoded with preselected agents will now be described. FIG. 2 illustrates a diffusion process for encoding (loading) apoferritin nanoparticles 10 with preselected agents 12. Here, the process is described in reference to metal phosphate. In the figure, apoferritin nanoparticle 10 again provides an inner cavity (core) 16. Metal ions 22 introduced in solution, e.g., at pH 8, first diffuse into apoferritin cavity (core) 16 through channels (not shown), and accumulate along the internal surface of the cavity. Phosphate buffer saline (PBS) containing phosphate (PO₄²⁻) ions 18, e.g., at pH 2, is slowly introduced into the solution. Precipitation of metal phosphate 12 first occurs along the inner surface of the cavity 18. Metal ions 22 and phosphate anions 18 outside cavity 16 continue to diffuse into the cavity because of concentration differences. Seeds of metal phosphate 12 in the cavity act as autocatalysts, promoting continued growth of the seeds. Precipitation continues with addition of phosphate buffer solution at pH 2 until apoferritin cavity 18 fills with metal phosphate 12. Release of encoded metal ions 22 is effected with a change in solution pH, e.g., to a pH of 4.6. While the encapsulation process (FIG. 1) and diffusion process (FIG. 2) for preparation of apoferritin nanoparticles have been described separately herein, processes are not limited to use of a single encapsulation or diffusion process. For example, apoferritin nanoparticles can be encoded with various preselected agents involving any of a combination of one or more encapsulation or diffusion steps. Thus, no limitations are intended by the description to individual processes.

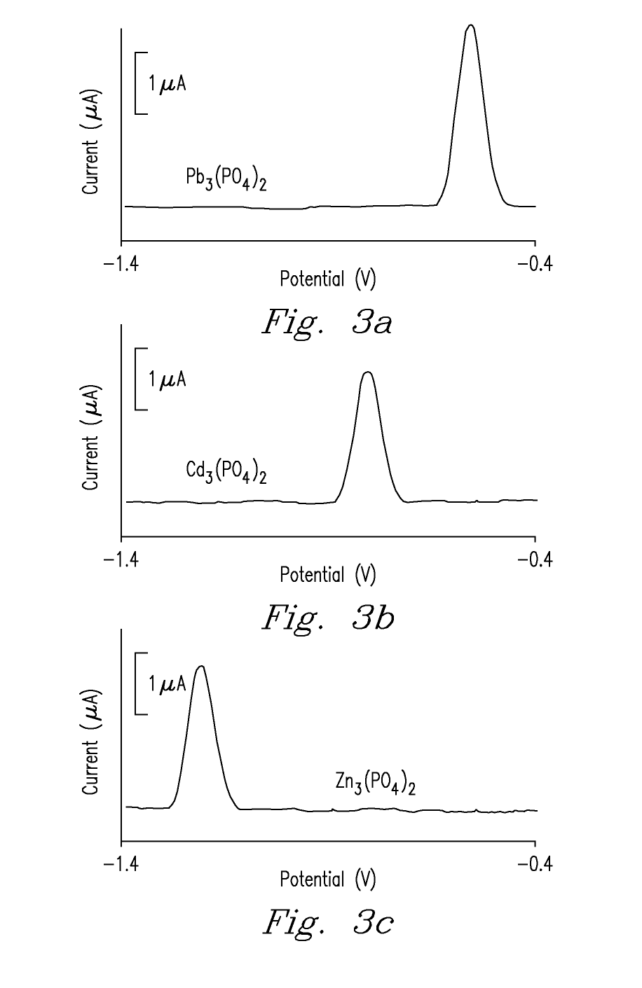

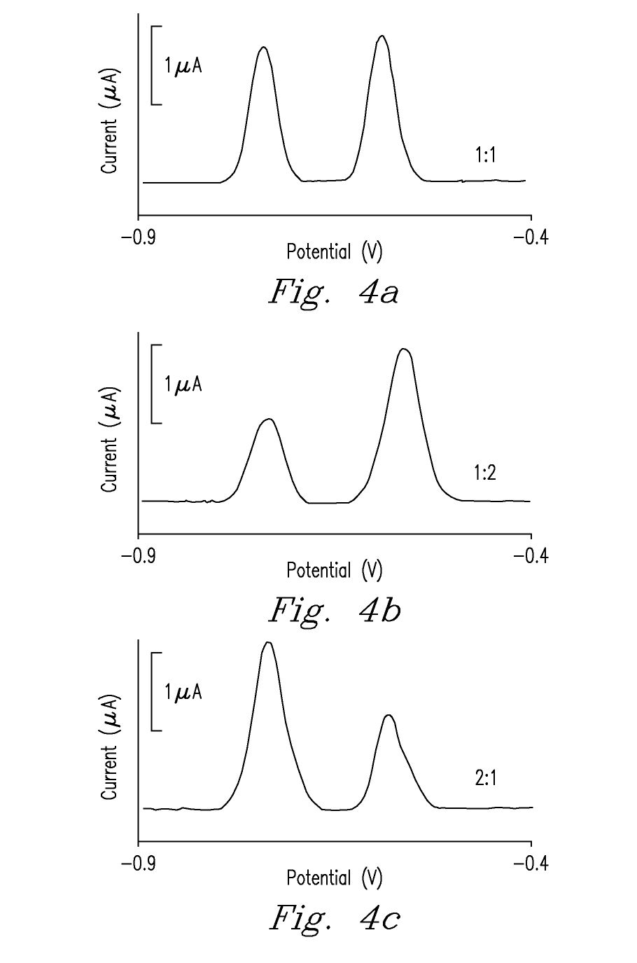

FIGS. 3a-3c present results from electrochemical measurements of apoferritin nanoparticles encoded within single metal phosphates, e.g., lead phosphate (FIG. 3a), cadmium phosphate (FIG. 3b), and zinc phosphate (FIG. 3c), respectively. In the figures, current (pA) is plotted as a function of potential (V) in typical square-wave voltammograms (SWVs). Results demonstrate that peak heights and peak resolution are sufficient for electrochemical detection and measurement of each metal encoded within the apoferritin nanoparticles. FIGS. 4a-4c present results from electrochemical measurements of apoferritin nanoparticles encoded with two preselected metal phosphates, i.e., cadmium (Cd) phosphate and lead (Pb) phosphate, with mole ratios for [Cd:Pb] of [1:1] (FIG. 4a), [1:2] (FIG. 4b), and [2:1] (FIG. 4c), respectively. In the figures, current (μA) is plotted as a function of potential (V) in typical square-wave voltammograms (SWVs). Results show peak height and peak resolution are sufficient for measurement of each of the two preselected metals encoded within the apoferritin nanoparticles. In addition, results further demonstrate that concentration of each encoded agent can be varied for a desired measurement outcome. While applicability of processes disclosed herein have been demonstrated for apoferritin nanoparticles encoded with single and dual metal phosphates, the methods are not limited thereto. For example, electrochemical detection can currently measure at least five to six metals simultaneously in a single measurement with minimal peak overlap. Thus, apoferritin nanoparticles encoded with, e.g., five or six different metal phosphates at, e.g., five or six different concentrations or ratios would provide thousands of usable voltammetric signatures that can be expected to be applicable in a wide variety of sensor, device, and/or assay applications. Exemplary applications are described further herein. Functionalization of the surface of apoferritin nanoparticles will now be described.

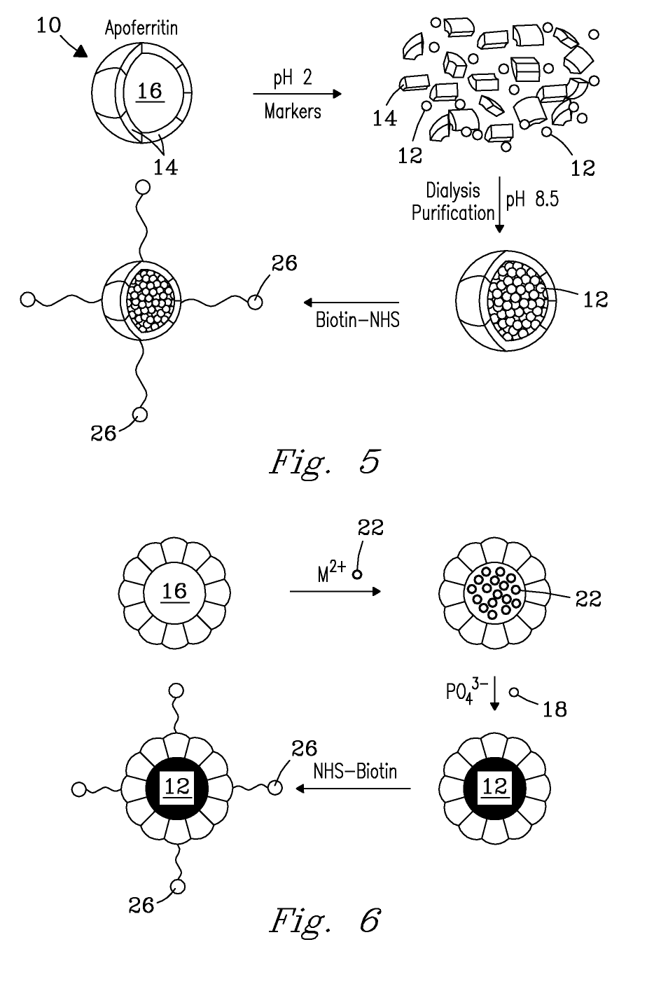

FIG. 5 and FIG. 6 illustrate processes for functionalization of an outer surface of apoferritin nanoparticles, which nanoparticles are encoded by the method of encapsulation and diffusion, respectively, described previously herein. In these figures, apoferritin nanoparticles 10 encoded (loaded) with preselected agents 12 are subsequently functionalized with preselected biomolecules and chemical constituents. In these figures, the preselected agent is represented by a metal phosphate 12, where the metal is introduced as metal cations 22, described previously herein. Amino acid residues present at the end of channels (not shown) of apoferritin nanoparticles 10 provide a facile route for attaching various biomolecules 26 or chemical constituents 26 to an outer surface of the apoferritin nanoparticles. Biotin is an exemplary molecule for functionalizing surfaces of encoded apoferrin nanoparticles, which can be attached to the surface with a biotinylation reagent, e.g., biotinamidohexanoyl-6-amino-hexanoic acid N-hydroxy-succinimide (NHS) ester (biotin-NHS). Functionalization of the nanoparticle surface can be prior to, or following, the encoding of the apoferritin nanoparticle with preselected agents, e.g., as described previously in reference to FIGS. 1 and 2. Separation of the functionalized apoferritin nanoparticles from the preparation medium can be achieved by conventional methods. Functionalized and encoded nanoparticles can be used, e.g., as biochemical tags (biochemical labels) in biosensor, protein assay, and immunoassay applications described further herein. While the functionalization process is described here with reference to biotin, the invention is not limited thereto. As described further herein, nanoparticles can be functionalized with various surface groups and molecules including, but not limited to, e.g., proteins, antibodies, antigens, vitamins and cofactors. All surface groups, constituents, and molecules as will be selected by those of skill in the biochemical arts are within the scope of the present invention. No limitations are intended.

### Assay and Immunoassay Applications for Functionalized Encoded Nanoparticles

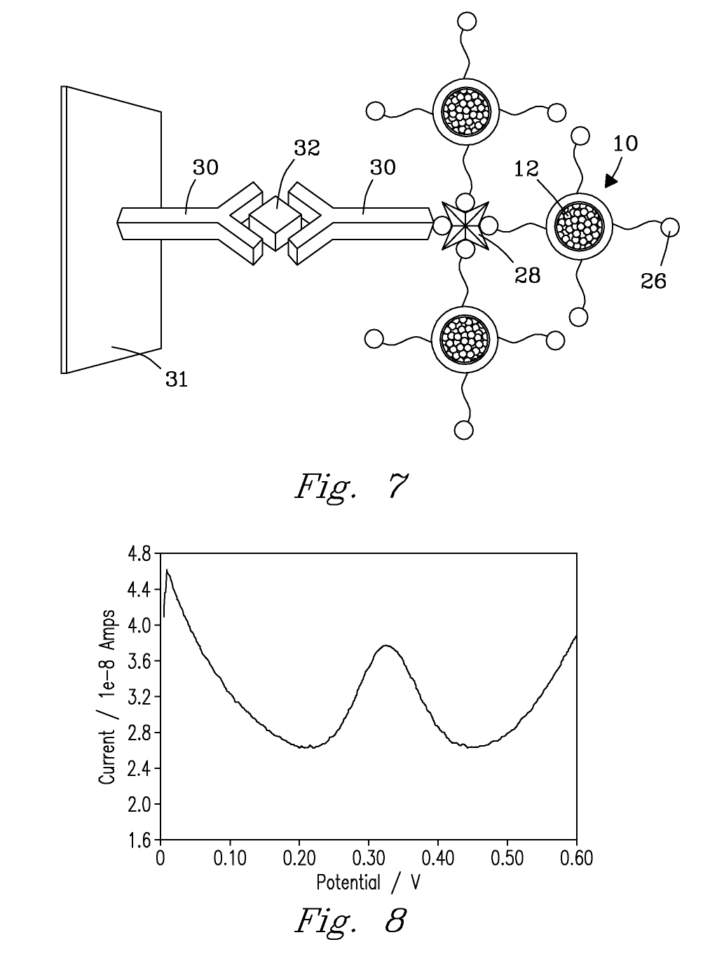

FIG. 7 illustrates a simple immunoassay protocol involving fluorescence marker encoded apoferritin nanoparticles that find use, e.g., as biochemical labels (i.e., fluorescent labels). Fluorescein (C₂₀H₁₂O₅) (CAS No. 2321-07-5] is an exemplary fluorescence marker (agent) (i.e., fluorophore) containing functional groups that absorb energy at specific wavelengths and re-emit the wavelengths (i.e., Fluoresce) at a different wavelength. Fluorescein has an absorption maximum at 494 nm and an emission maximum of 521 nm (in water). Fluorescein also has absorption for all solution pH values at 460 nm, useful in quantitative analysis. Fluorescein isothiocyanate (FITC) (molecular formula: C₂₁H₁₁N05S) [CAS No. 27072-45-3], a chemical derivative of fluorescein, is another exemplary fluorescence agent described further herein that finds use, e.g., as a fluorescence marker or tracer in, e.g., biochemical labels, e.g., in conjunction with apoferritin nanoparticles described herein. Fluorescein and fluorescein isothiocyanate (FITC) are exemplary, but not exclusive, fluorescence agents for encoding (loading) of functionalized apoferritin nanoparticles described herein. No limitations are intended. In the figure, the immunoassay employs the following components and elements: a suitable stationary phase 31 (e.g., an aldehyde-modified, aminosilanated glass slide, magnetic beads, or other stationary phase); a primary antibody 30 (e.g., anti-mouse IgG) immobilized on stationary phase 31; an antigen 32 (e.g., mouse IgG); a biotin-functionalized secondary antibody 30 (e.g., anti IgG with different epitopes); a bridging protein (linker) 28 (e.g., streptavidin, avidin, etc.); and encoded apoferritin nanoparticles that are functionalized with biotin 26. In the instant example, the immunoassay involves antigen 32 (e.g., mouse IgG), which binds to a target (or capture) antibody 30 (e.g., anti-mouse IgG). Antigen 32 binds with the biotin-modified 26 secondary antibody 30. Biotin-modified secondary antibody 30 then selectively binds to a preselected bridging protein (linker) 28 (e.g., streptavidin), that provides up to four binding sites that couple with biotin-functionalized, fluorescein-encoded apoferritin nanoparticles 10 in a sandwich type immunocomplex. Here, streptavidin, a surface protein, provides four binding sites that can bind with: 1) biotin functionalized nanoparticles; 2) a biotin-modified secondary antibody; and up to three sites that bind with biotin-modified apoferritin nanoparticles as immunoassay labels. Here, the sandwich complex in the immunoassay can be measured and quantified, e.g., by fluorescence microscopy. In particular, presence of nanoparticles encoded with a fluorescence marker in the sandwich immunocomplex allows for measurement of antigen binding events in the immunoassay. In the instant application, measurement sensitivity of the fluorescein-loaded apoferritin nanoparticles (labels) was 125 times greater than for single fluorescein labeled antibody (anti-mouse IgG) controls. Signal enhancement is attributed to: a maximum loading of, e.g., fluorescein in the core of the apoferritin nanoparticles (˜65 fluorescein anions per nanoparticle) and four binding sites of the avidin linker that provide a maximum number of nanoparticle labels. While the immunoassay binding events and functionalization of apoferritin nanoparticles are described herein with reference to use of exemplary biotin/streptavidin interactions, the invention is not limited thereto. Those of skill in the biochemical and immunological arts will understand that various immunocomplexes and immunoconjugates can be constructed using a variety of biochemical interactions, e.g., bispecific antibody-hapten-peptide—interactions. Thus, no limitations are intended. All immunocomplex and biochemical interactions as will be contemplated by those of skill in the art in view of the disclosure are within the scope of the invention.

In another exemplary bioassay and immunoassay application, biotin-functionalized apoferritin nanoparticles were encoded with hexacyanoferrate (II) (tetra-sodium salt) [molecular formula: C₆FeN₆Na₄ [CAS No. 13601-19-9] or hexacyanoferrate (III) [molecular formula: FeK₃(CN)₆ [CAS No. 13746-66-2] as an electrochemical (or redox) marker. Briefly, a solution containing the biotin-functionalized, hexacyanoferrate encoded apoferritin nanoparticles were incubated with an avidin-coated glass slide prepared with an immobilized (anti-mouse IgG) antibody and target (mouse IgG) antigen as described above (see FIG. 7) for fluorescein-encoded apoferritin nanoparticles, with avidin as a bridging protein. FIG. 8 shows a typical square wave voltammogram obtained in the electrochemical measurement of hexacyanoferrate released from biotin-modified encoded apoferritin nanoparticles, which deliver a measureable electrochemical (voltaic) signal suitable for electrochemical and/or immunoassay applications.

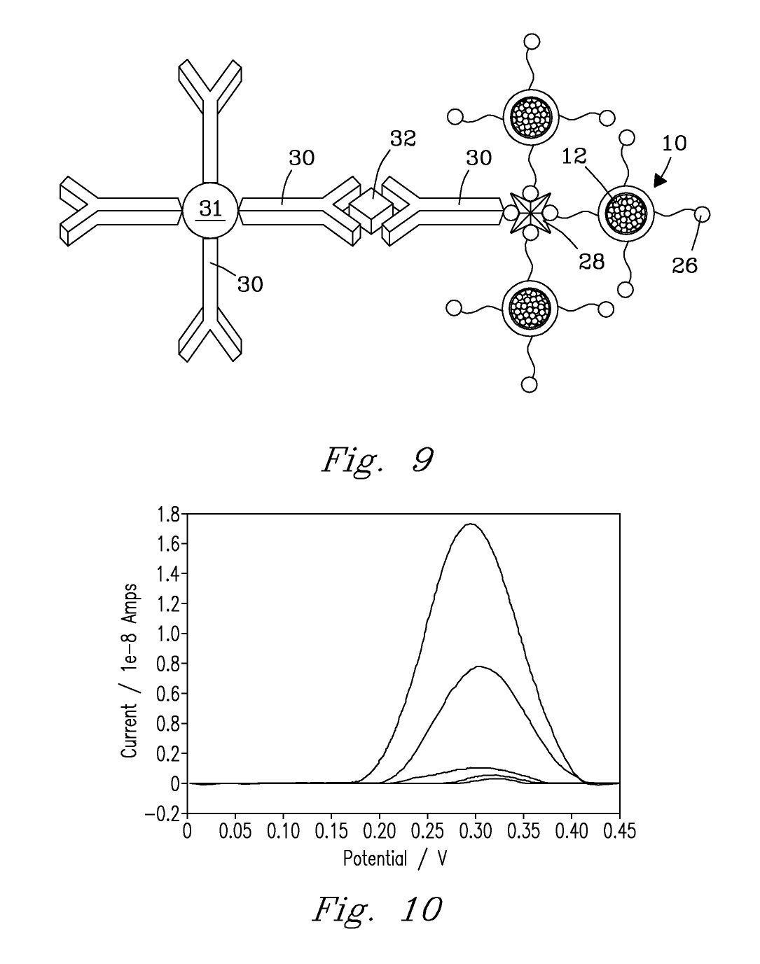

FIG. 9 illustrates another exemplary sandwich immunoassay protocol involving hexacyanoferrate encoded nanoparticles as a suitability test for electrochemical immunoassay applications. The (MB)-based sandwich immunoassay protocol includes the following components: magnetic beads 31 (as the target stationary phase) with antibodies 30 (e.g., anti-IgG) attached at the surface, an antigen 32 (e.g., IgG), a secondary antibody 30 (e.g., anti-IgG) modified to include biotin 26, a bridging (linker) protein 28 (e.g., streptavidin, avidin, etc.), and the hexacyanoferrate 12-loaded and biotin 26-modified apoferritin-nanoparticles 10. In the figure, two immunoreactions occur between: 1) a primary antibody 30 (anti-IgG) linked to magnetic beads 31 and 2) a biotin-modified secondary antibody 30 (e.g., e.g., anti-IgG) in the presence of antigen (IgG) 32, described previously in reference to FIG. 7. Introduction of a bridging protein 28, e.g., streptavidin, binds the hexacyanoferrate-loaded biotin-modified apoferritin nanoparticles 10. In the immunoassay, release of encoded hexacyanoferrate 12 from the apoferritin nanoparticles 10, permits electrochemical detection of the immunological assay event, which here quantifies binding of the target antigen 32 (e.g., mouse IgG). FIG. 10 presents results obtained in the electrochemical measurement of the hexacyanoferrate encoded, biotin-functionalized apoferritin nanoparticles as a function of increasing concentration of target antigen (IgG), i.e., from 0.1 ng/mL IgG. In the figures, current (pA) is plotted as a function of potential (V) in typical square wave voltammograms, which show hexacyanoferrate encoded nanoparticles deliver measureable electrochemical signals suitable for sensitive electrochemical detection in immunoassay applications.

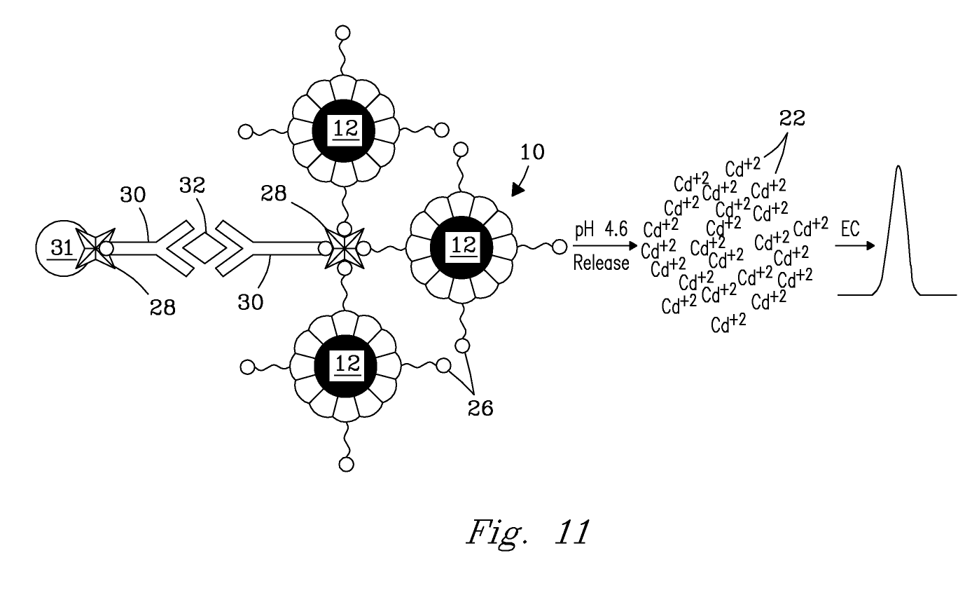

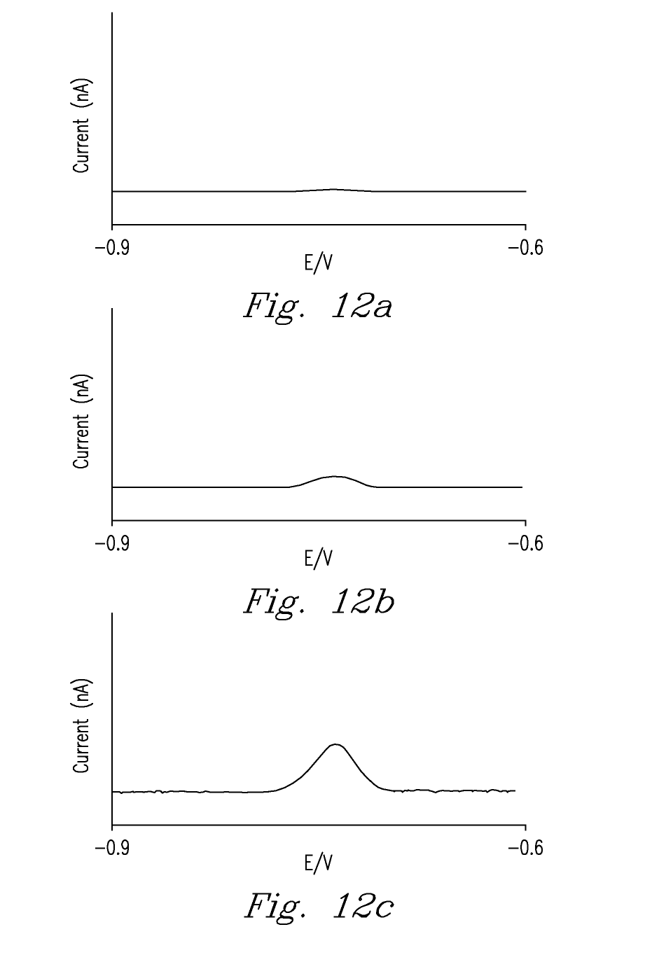

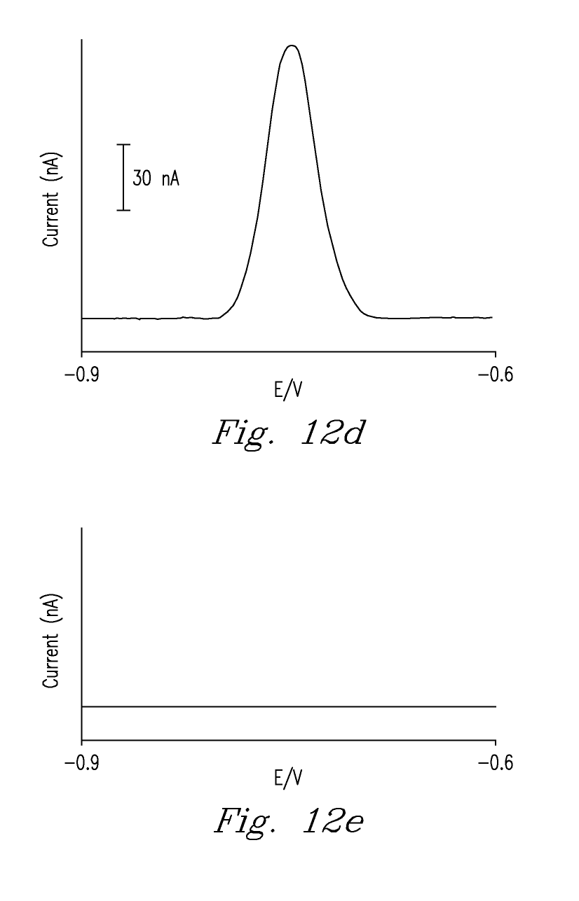

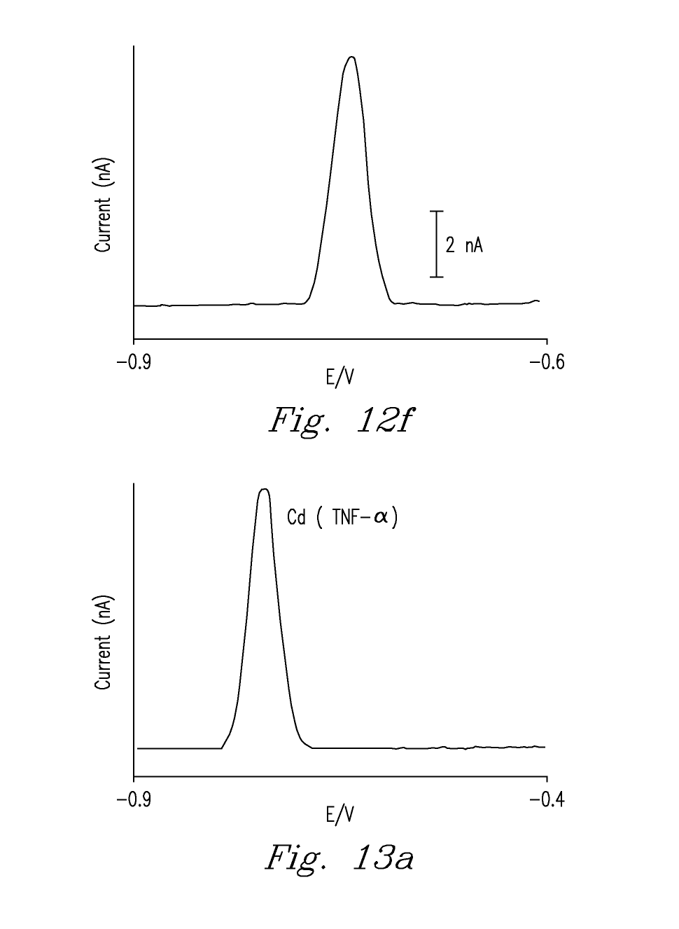

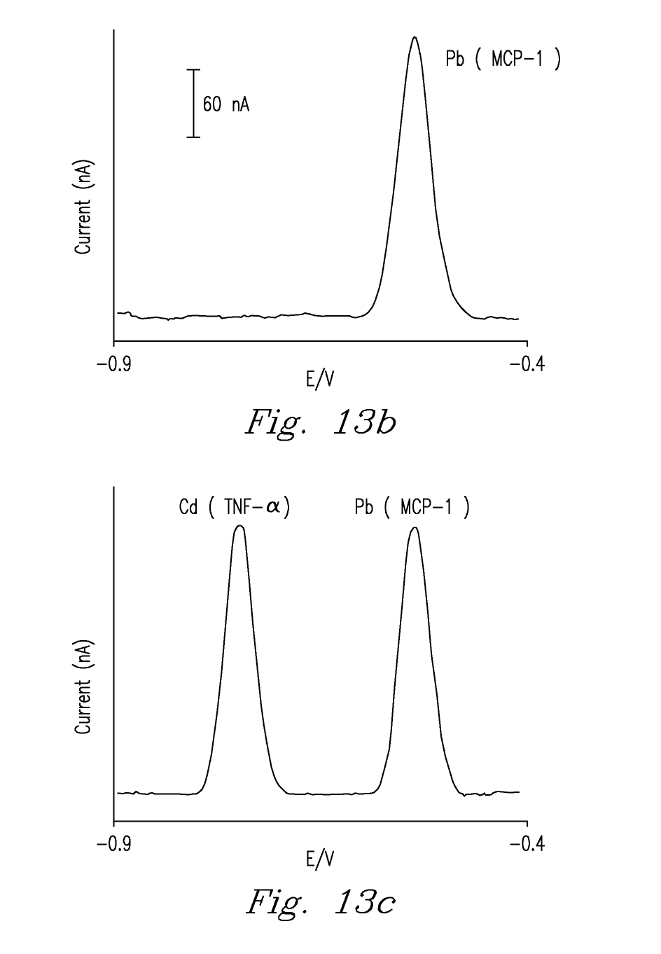

FIG. 11 is a schematic showing another exemplary sandwich immunoassay protocol involving single metal phosphate encoded nanoparticles for electrochemical immunoassay applications, e.g., for bioassay for cancer detection. Here, the protocol includes: magnetic beads 31 as a support surface, modified to include an antibody 30 (e.g., anti-tumor necrosis factor (anti-TNF-α) antibody); an antigen 32 [e.g., tumor necrosis factor (TNF-α) antigen]; a secondary antibody 30 (e.g., anti-TNF-α antibody) modified to include biotin 26; a bridging (linker) protein 28 (e.g., streptavidin); and apoferritin nanoparticles 10 encoded with metal phosphate 12 as a preselected agent 12. In the instant test, cadmium phosphate was used as the metal phosphate. Release of cadmium ions 22 from the encoded nanoparticles in acetate buffer at pH 4.6 provides for electrochemical detection, measurement, and quantification of positive immunoassay events. FIGS. 12a-12f present typical square wave voltammograms obtained from sandwich immunoassays described previously in reference to FIG. 11 as a function of increasing concentrations of (TNF-α) target antigen: 0 ng/mL (FIG. 12a); 0.1 ng/mL (FIG. 12b); 1 ng/mL (FIG. 12c); 10 ng/mL (FIG. 12d); and 0.01 ng/mL (FIG. 12f). Cadmium released from encoded biotin-modified nanoparticles in the sandwich complexes of the immunoassay provide for electrochemical measurement of these immunoassay events. As shown in the figures, measureable electrochemical signals are generated upon release of cadmium ions from the encoded nanoparticles over a linear detection range as a function of increasing concentration of target (i.e., tumor necrosis factor, TNF-α) antigen. At a concentration of 10 pg/mL TNF-α target antigen (FIG. 12f), a detection limit of about 2 pg/mL (77 fM), or about 2.33×10⁶ TNF-α biomarker molecules, was obtained. No significant signal was observed in the absence of TNF-α target antigen (FIG. 12a), or in cases of large excesses (˜1000-fold) of non-specific protein [i.e., macrophage chemotactic protein-1, MCP-1] biomarkers that do not bind to (anti-TNF-α) antibody in the immunoassay (FIG. 12e). Results demonstrate the suitability of functionalized, single metal phosphate encoded nanoparticles for sensitive, quantitative electrochemical detection and measurement of positive immunoassay events in immunoassay applications with excellent selectivity and high reproducibility. FIGS. 13a-13c show typical square wave voltammograms obtained from electrochemical measurement of sandwich immunoassay complexes (i.e., magnetic bead-antibody-antigen-biotin modified antibody coupled to nanoparticles encoded with metal phosphate) described previously in reference to FIG. 11. In these tests, apoferritin nanoparticles encoded with single metal phosphates (e.g., cadmium phosphate and lead phosphate) were modified with two different antibodies (anti-TNF-α antibody and anti-MCP-1 antibody) as biomarkers, respectively, for detection of target antigens (TNF-α and MCP-1). As illustrated in the figures, individual biomarkers yielded well-defined and resolved peaks with similar sensitivity at −0.73V for (10 ng/mL) TNF-α target antigen (FIG. 13a) measured with cadmium encoded nanoparticles, and −0.55 V for (10 ng/mL) MCP-1 target antigen (FIG. 13b) measured with lead encoded nanoparticles. A sample mixture containing both TNF-α and MCP-1 target antigens (10 ng/mL) shows two well-resolved signals at similar potentials (FIG. 13c). Results demonstrate that both single and dual metal phosphate encoded apoferritin nanoparticles yield well-resolved voltammetric signals suitable for electrochemical detection of various and multiple biomarkers in assay and immunoassay applications. For example, assays that involve these metal encoded nanoparticles are ultrasensitive, with detection limits as low as 77 fM. Further, simultaneous detection of multiple target antigens has been demonstrated using nanoparticles encoded with at least two metal phosphates (e.g., cadmium phosphate and lead phosphate). The functionalized apoferritin nanoparticles described herein have potential applications in electrochemical biosensors and bioassays, e.g., for detection of DNA and proteins; for immunoassays, and other related applications. While the instant immunoassay applications have been illustrated and described in reference to functionalized apoferritin nanoparticles encoded with single and dual metal phosphates, the invention is not limited thereto. For example, other metal phosphates [including, e.g., zinc (Zn), lead (Pb), cadmium (Cd), copper (Cu), indium (In), gold (Au), and silver (Ag)], metal sulfides, and metal sulfates can be encoded. Further, number of encoded metals is not limited. For example, apoferritin nanoparticles can be encoded with two or more metal phosphates, sulfides, and sulfates, including predetermined concentrations and/or ratios of mixed metal phosphates, sulfides, and sulfates that provide a wide range of distinguishable and uniquely identifiable electrical signatures for electrochemical detection, e.g., for protein biomarkers in various assay and sensor applications. Thus, no limitations are intended by descriptions to exemplary metal phosphates (and metal sulfides and metal sulfates).

### Radioimmunodetection and Radioimmunotherapy

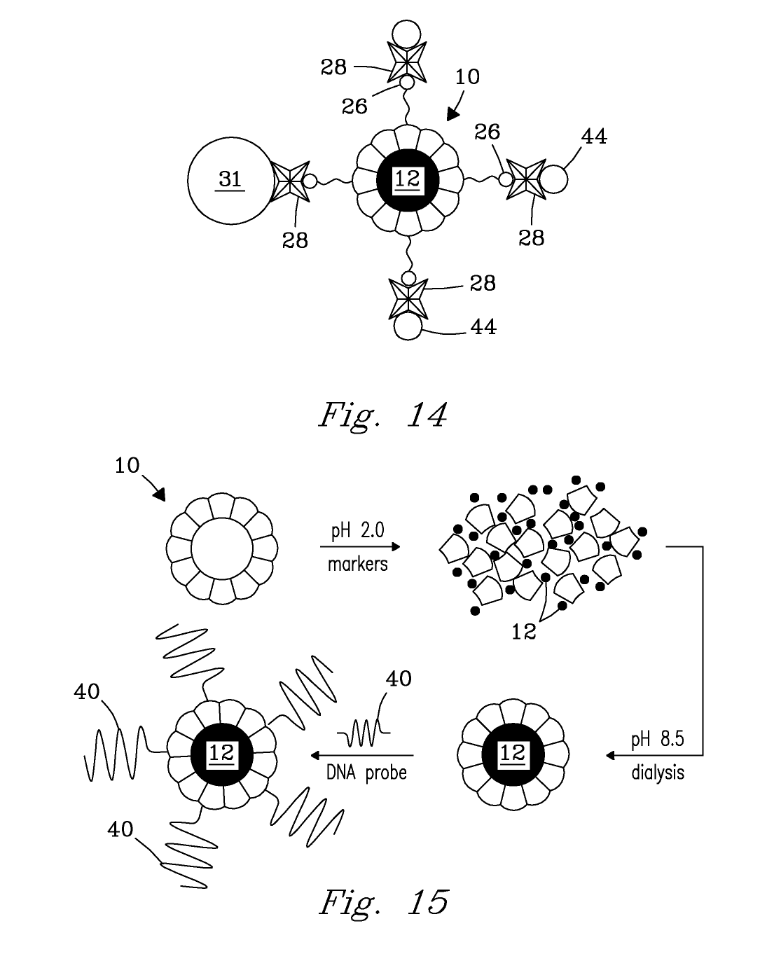

Surface functionalized apoferritin nanoparticles internally encoded with diagnostic and radiotherapeutic agents, e.g., radioisotopes, will now be described, suitable for radioimmunodetection, radioimmunoimaging, and radioimmunotherapy. FIG. 14 illustrates a functionalized apoferritin nanoparticle 10 encoded with lutetium phosphate as the preselected agent 12. Here, elemental lutetium, a non-radioactive mixture of the stable isotopes, i.e., lutetium-175 (97.4%) and lutetium-176 (2.6%) was used as a surrogate for the radioisotope lutetium-177 (¹⁷⁷Lu). Lutetium-177 is a potentially useful radioisotope that has applications for radioimmundetection and radioimmunotherapy of various cancers. For example, in a biological host (e.g., human body) at a pH of from 7 to 8, the lutetium phosphate core of the apoferritin nanoparticle encoded with the radioisotope lutetium-177 (¹⁷⁷Lu) is insoluble, which makes the resulting nanoparticle (when coupled to proteins such as streptavidin that target cell surface antigen binding sites) ideally suited for diagnosis and/or for treatment of cancers. Lutetium phosphate is exemplary of many other suitable radioisotopes that can be encoded into apoferritin nanoparticles, e.g., as phosphates, sulfides, or sulfates. Lutetium (III) cations easily diffuse into the inner core of the apoferritin nanoparticles through hydrophilic channels, as detailed previously herein, which at pH 8.0, have a negative electrostatic potential that facilitates diffusion of the lutetium cations into the apoferritin core. Functional groups (e.g., carbonate or phosphate) on the inner surface of the cavity function as chelating groups that facilitate the concentration of the isotope within the apoferritin cavity. Maximum loading of lutetium in the apoferritin cavity is attained by optimization of parameters including, but not limited to, e.g., metal cation and counter ion concentrations (e.g., phosphate or sulfate), pH, and diffusion time. The encoded apoferritin nanoparticle is subsequently functionalized, e.g., with biotin 26 which binds selectively with, e.g., a bridging protein 28 such as avidin or streptavidin, that then can selectively bind with antigens on the surface of tumor cells. Here, streptavidin acts as a bridge with biotin for binding in the immunoassay complex. Conjugation of biotin to the surface of the apoferritin nanoparticle is achieved by incubating the lutetium phosphate encoded apoferritin nanoparticles with a biotin-NHS reagent and removing excess biotin-NHS. Amino groups at the end of the apoferritin nanoparticle channels conjugate with biotin and provide a facile route to biotinylation of the surface. In the figure, magnetic bead 31 is representative of tumor cell interaction. Here, magnetic bead 31 has a surface modified with a streptavidin molecule 28 that can bind with at least one biotin 26 at the surface of the biotin-modified (biotinylated) nanoparticles 10 encoded with lutetium phosphate as the preselected agent 12. The apoferritin nanoparticle attaches to the streptavidin 28 functionalized magnetic bead 31 via another streptavidin/biotin conjugation reaction. By attaching, e.g., a fluorescence marker 44 (e.g., fluorescein isothiocyanate, FITC), the pseudo-pretargeting event (i.e., complex comprising the MB/biotin modified lutetium phosphate encoded apoferritin nanoparticle/FITC marker) can be measured by detection of the fluorescence marker. While the process has been illustrated and described with reference to the encoding of lutetium as a (surrogate) radioisotope, the process is not limited thereto. For example, the process has also been demonstrated using apoferritin nanoparticles encoded with yttrium-89 (⁸⁹Y), the only stable isotope of elemental yttrium, a nonradioactive surrogate of the radioisotope yttrium-90 (⁹⁰Y). Yttrium-90 has a physical half-life of 64 hours which is suitable for radioimmunotherapy of various cancers. As another example, the radioisotope Indium-111 (¹¹¹In), has a physical half-life of 67 hours and is often paired with yttrium-90 in radioimmunotherapy applications because indium-111 (¹¹¹In), photon emissions are detectable by nuclear medicine imaging systems (e.g., gamma probes and cameras), whereas yttrium-90 is a pure beta emitter that does not emit photons for imaging. Gamma cameras image radioisotopes that emit photons with gamma energies of between about 80 keV and about 450 keV. Radioisotopes suitable for use with gamma probes and cameras include, but are not limited to, e.g., copper-67 (⁶⁷Cu), lutetium-177 (¹⁷⁷Lu); rhenium-186 (¹⁸⁶Rh); rhenium-188 (¹⁸⁸Rh); technetium-99m (⁹⁹mTc); indium-111 (¹¹¹In); gadolinium-153 (¹⁵³Gd); and including combinations of these radioisotopes. Positron emission (PET) imaging instruments image radioisotopes that emit positrons with energies of 511 keV. Radioisotopes suitable for use with positron emission (PET) imaging instruments include, but are not limited to, e.g., copper-64 (⁶⁴Cu), gallium-68 (⁶⁸Ga); rubidium-82 (⁸²Rb); bromine-77 (⁷⁷Br); zirconium-89 (⁸⁹Zr); arsenic-71 (⁷¹As); arsenic-72 (⁷²As); arsenic-74 (⁷⁴As); yttrium-86 (⁸⁶Y); yttrium-88 (⁸⁸Y); and iodine-124 (¹²⁴I); and, including combinations of these radioisotopes. Radioisotopes suitable for radiotherapy include, but are not limited to, e.g., radium-223 (²²³Ra); yttrium-90 (⁹⁰Y); lutetium-177 (¹⁷⁷Lu); phosphorus-32 (³²P); phosphorus-33 (³³P); iodine-131 (¹³¹I); astatine-211 (²¹¹At); bismuth-212 (²¹²Bi); bismuth-213 (²¹³ Bi); lead-212 (²¹² Pb); actinium-225 (²²⁵Ac); holmium-166 (¹⁶⁶Ho); samarium-153 (¹⁵³Sm); and, including combinations of these radioisotopes. Other radioisotopes are anticipated to follow similar preparation and reaction pathways for uses in radioimmunotherapy and radioimmundetection of various cancers. Thus, no limitations are intended. All radioisotopes for detection and treatment of diseases as will be selected by those of skill in the art in view of the disclosure are within the scope of the invention. In general, pre-biotinylated apoferritin as a synthesis template reduces nanoparticle preparation times. Loading capacity of pre-biotinylated apoferritin is similar to that for non-biotinylated apoferritin. And, the biotinylation process does not appear to block diffusion of lutetium and yttrium cations, as well as other metal cations, and/or phosphate anions into the apoferritin cavity. And, pre-biotinylated apoferritin as a template significantly promotes metal loading capacity, e.g., with 360 yttrium atoms per apoferritin molecule. Application of pre-biotinylated apoferritin as the template in synthesis of radioisotope encoded metal phosphates can shorten preparation times, allowing more radioactive atoms to be available for therapy. The person of skill in the art will realize that many and varied surface modifiers can be employed in conjunction with the processes and complexes and applications described herein. Surfaces of the various assay and immunoassay components including, e.g., the apoferritin nanoparticles, can be functionalized with other molecules including, e.g., proteins, antibodies, antigens, nucleic acid, nucleotides, specific biomarkers, detection agents, labels, and tags, and other suitable constituents as will be known by those in the biochemical and immunological arts that provide functionality to the apoferritin nanoparticles for use as biosensory tags, labels, and detection probes. Bioassay applications involving apoferritin nanoparticles encoded with preselected markers and agents including, e.g., assays (e.g., DNA and protein assays) and immunoassays have been demonstrated, described hereafter. DNA functionalized apoferritin nanoparticles encoded with preselected agents (e.g., fluorescence agents) will now be described.

FIG. 15 illustrates a protocol for preparation of DNA functionalized apoferritin nanoparticles 10 encoded with preselected agents 12. Here, apoferritin nanoparticles are encoded with, e.g., hexacyanoferrate (III) and fluorescein as electrochemical and fluorescence labels, respectively, that provide for electrochemical or spectral detection in the intended assay. Briefly, apoferritin nanoparticle 10 disassembles into subunits 14 at pH 2 in the presence of one or more preselected agents 12. Apoferritin nanoparticle 10 reassembles (described in reference to FIG. 1) at pH 8.5 encoding the preselected agent within the cavity (core) of the nanoparticle. Surface of the apoferritin nanoparticles can be functionalized, e.g., with an amino-modified DNA probe 40, using a coupling reagent, e.g., 1-ethyl-3-(dimethylaminopropyl)carbodiimide hydrochloride (EDC) to obtain the DNA functionalized apoferritin nanoparticle 10. For hexacyanoferrate encoded nanoparticles, about eight DNA probes are attached per nanoparticle. Results have demonstrated that the core of the encoded apoferritin nanoparticle does not change spectral characteristics of the fluorescence or electrochemical markers.

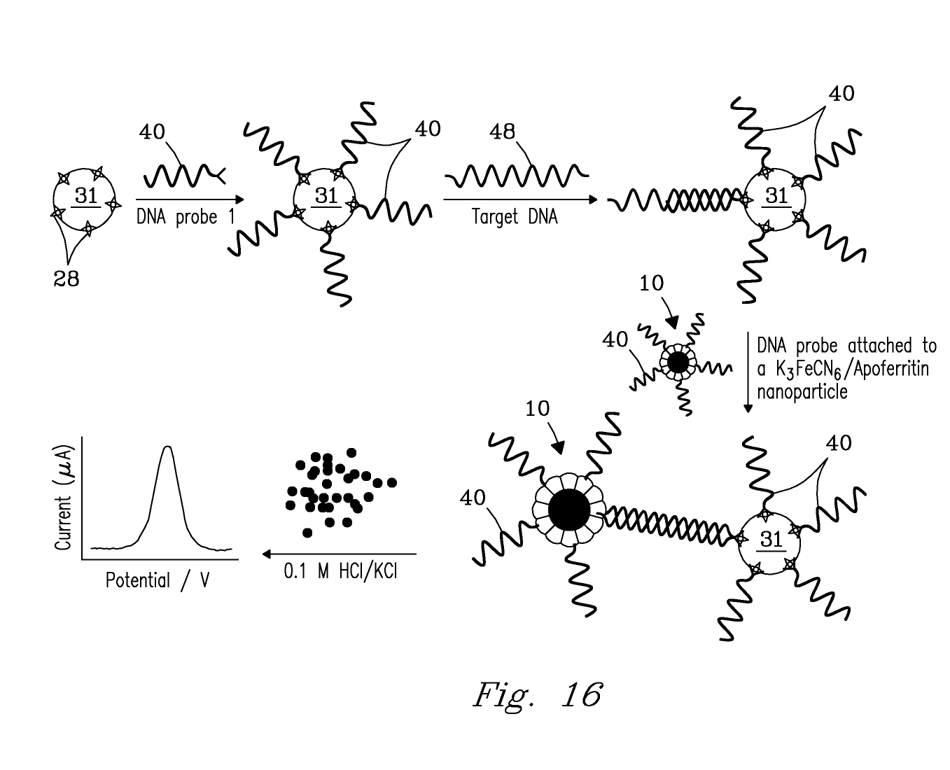

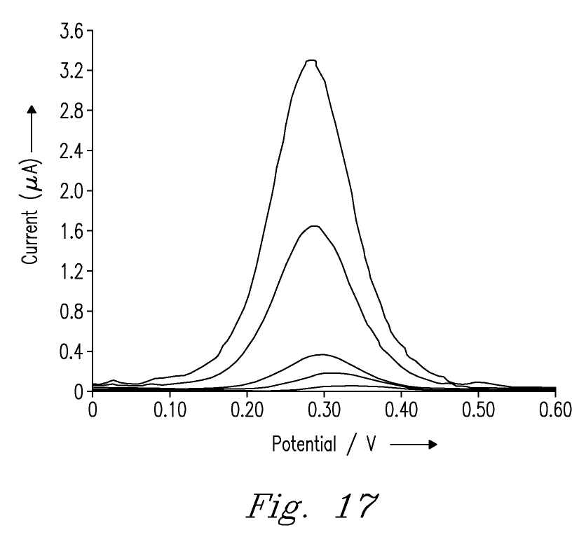

FIG. 16 presents a protocol for use of DNA functionalized, encoded apoferritin nanoparticles 10, e.g., as a label for quantitative electrochemical detection and assay of a target DNA 48, i.e., single-nucleotide polymorphisms (SNPs) 48, described further herein. The process involves a dual hybridization event. In the figure, a streptavidin 28-modified magnetic bead 31 is further modified at the surface to include a DNA probe 40. DNA probe 40 attached to the MB 31 binds (hybridizes) with target DNA 48 in a first hybridization reaction. The target DNAs on the magnetic bead couple in a 2^{nd} hybridization reaction with apoferritin nanoparticles 10 functionalized with DNA probes 40. Here, nanoparticles, encoded with, e.g., hexacyanoferrate, act as labels for the electrochemical detection of target DNA. Release of hexacyanoferrate from the encoded nanoparticles (e.g., with 0.1M HCl/KCl) permits voltammetric detection. FIG. 17 presents a series of square wave voltammograms showing electrochemical measurements of hexacyanoferrate released from encoded apoferritin nanoparticles in the assay as a function of increasing concentration (i.e., 10, 50, 100, 500, and 1000 ng/L, respectively) of target DNA. Results show assays involving encoded apoferritin nanoparticles provide for ultra-trace (ng/L) measurements of target DNA. Nonspecific binding effects are insignificant. Limit of detection for these exemplary bioassays are approximately 3 ng/L (or 460 fm, based on a signal-to-noise ratio (S/N) of 3).

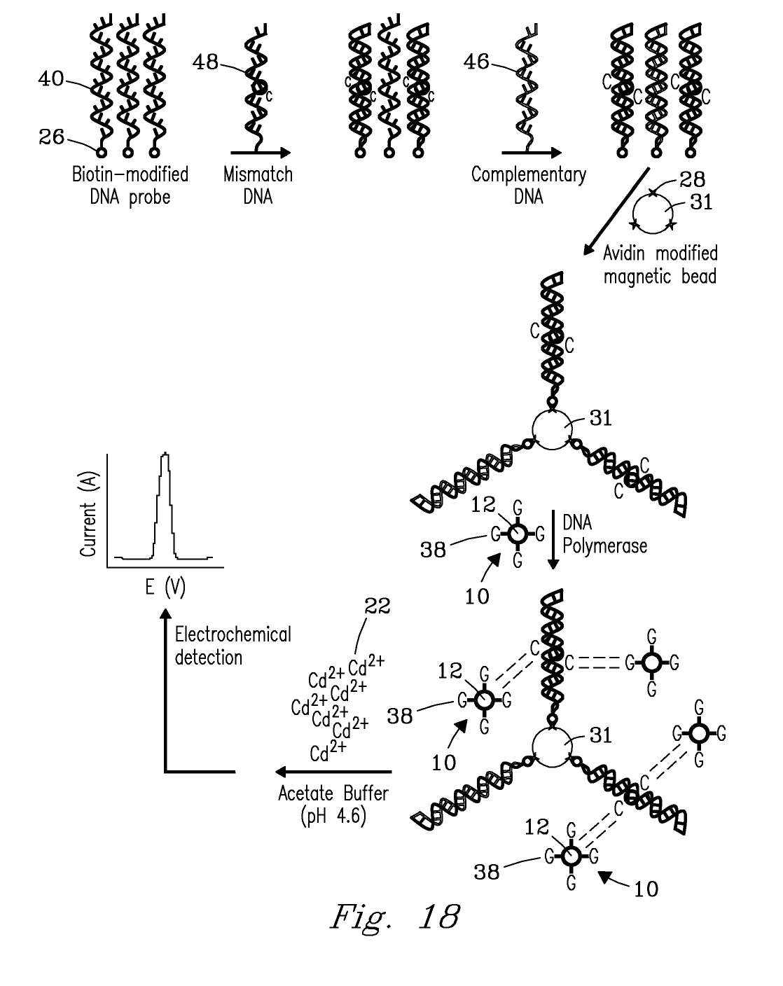

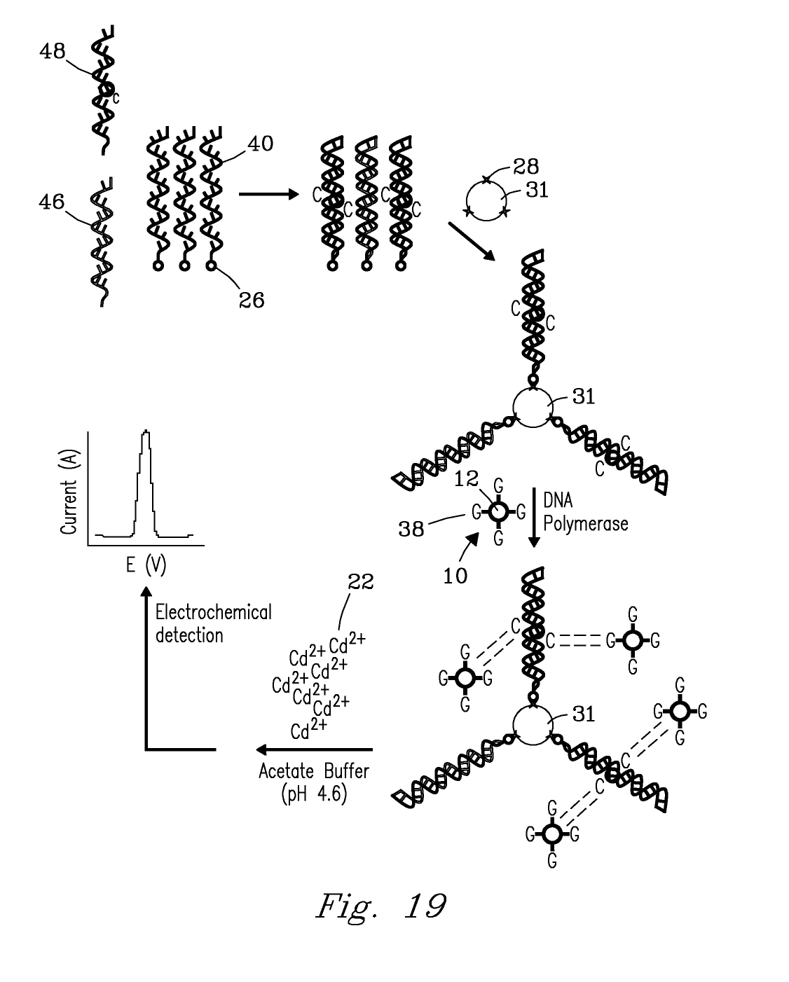

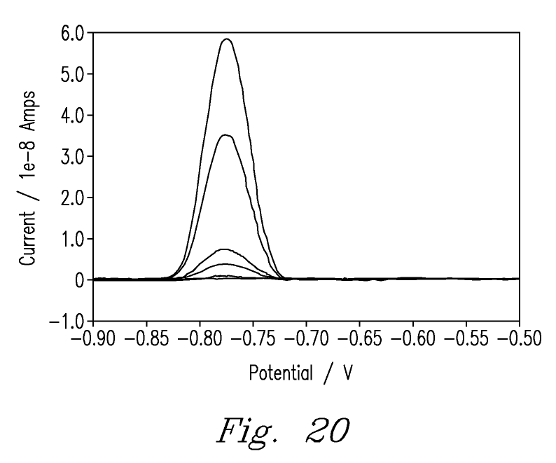

FIG. 18 illustrates another protocol employing nucleotide 38 functionalized apoferritin nanoparticles 10 encoded with metal phosphate 12 (e.g., cadmium phosphate) as preselected agent 12, for quantitative electrochemical detection and assay of single-nucleotide polymorphisms (SNPs) 48. Single-nucleotide polymorphisms 48 (also termed “mutant” or “mismatched” DNA) are DNA strands that have a single base pair nucleotide mismatch (a mutant site) within the duplexed DNA. In the figure, biotin 26 modified DNA probes 40 (i.e., biotinylated DNA probes), hybridized with mismatched DNA 48 and complementary DNA 46, are attached to the surface of avidin 28 modified magnetic beads 31 through a biotin-avidin conjugation reaction. Here, apoferritin nanoparticles 10 are functionalized with nucleotide 38, e.g., guanine 38. Guanine is complementary to the mutant site of the mutant DNA 48. Coupling of the nanoparticle is effected using a DNA polymerase (e.g., DNA polymerase I), which attaches the guanine modified nanoparticles to the mutant sites in the duplexed mutant DNA strands under standard base pairing. Release of cadmium ions 22 from the encoded apoferritin nanoparticles 10 attached to the mutant DNA sites in the duplexed mutant DNA strands, e.g., in acetate buffer at pH 4.6, allows for quantitative electrochemical determination of the mutant DNA. Exemplary tests detected 21.5 attomol of mutant DNA, sufficiently sensitive to provide quantitative analysis of nucleic acid without need for polymerase chain reaction preamplification. The method accurately determines SNPs at frequencies as low as 0.01. The protocol is expected to provide accurate, sensitive, rapid, and low-cost detection of SNPs. FIG. 19 shows a modified protocol to that illustrated in FIG. 18 that provides a one-step DNA hybridization reaction for detection of mutant DNA 48. In the figure, biotin 26-modified DNA probes 40 (i.e., biotinylated DNA probes) are first hybridized with mismatched (i.e., mutant) DNA 48 and complementary DNA 46 in a single step. Resulting duplex DNA helixes are attached to the surface of avidin 28-modified magnetic beads 31 through a biotin-avidin conjugation reaction, followed by magnetic separation. Again, nucleotide 38 (e.g., guanine) functionalized, cadmium phosphate encoded apoferritin nanoparticles 10 are coupled to mutant sites of mutant DNA strands 48. Release of cadmium ions 22 from the encoded apoferritin nanoparticles 10, e.g., in acetate buffer at pH 4.6, provides for quantitative electrochemical determination of mutant DNA. The electrochemical signal (current density) is proportional to the concentration of mismatched (or mutant) DNA concentration in the sample solution. In the instant protocol, it is necessary to block any excess biotin-modified DNA probes 40 to block cytosine sites of unhybridized DNA probes 40. This is done by adding complementary DNA 46, which then permits the SNPs 48 to be quantified. FIG. 20 presents electrochemical results for measurement of cadmium released from nucleotide encoded apoferritin nanoparticles in the assay as a function of increasing concentration (i.e., 0, 0.5, 2.5, 5.0, 25.0, and 50.0 picomoles/L) of the mismatched DNA target. Results show assays involving encoded apoferritin nanoparticles provide for ultratrace (ng/L) measurement of target DNA. Detection limit for this exemplary bioassay is estimated to be 3 ng/L (460 fm, based on S/N=3) in conjunction with a 60 min hybridization time. While the previous description has been directed to use of DNA and/or nucleotide functionalized, encoded apoferritin nanoparticles for assay of DNA and SNPs, applications are not limited thereto. Those of skill in the art will recognize that applications will depend: 1) on choice of preselected agents introduced to and encoded within the core of the apoferritin nanoparticles, and 2) choice of surface groups that functionalize the apoferritin nanoparticle. For example, various redox and optical makers can be encoded (loaded) into the cavity of the apoferritin nanoparticles for uses that include, e.g., optical and electrochemical bioassays. In addition, protocols described herein can be extended to capture small molecules, e.g., for drug delivery and other therapeutic applications. And, functionalized, encoded apoferritin nanoparticles described herein offer suitable methods for encoding and releasing markers and preselected agents of interest. Thus, various new nanoparticles are expected to be suitable for other biological assays and immunoassays. All modifications of functionalized, encoded nanoparticles as will be contemplated by those of skill in the art in view of the disclosure are within the scope of the invention. No limitations are intended to exemplary metals, surface modifiers, molecules, markers (redox, fluorescence, optical, etc.) and other preselected agents described herein. Further, no limitations are intended by description of exemplary applications described herein.

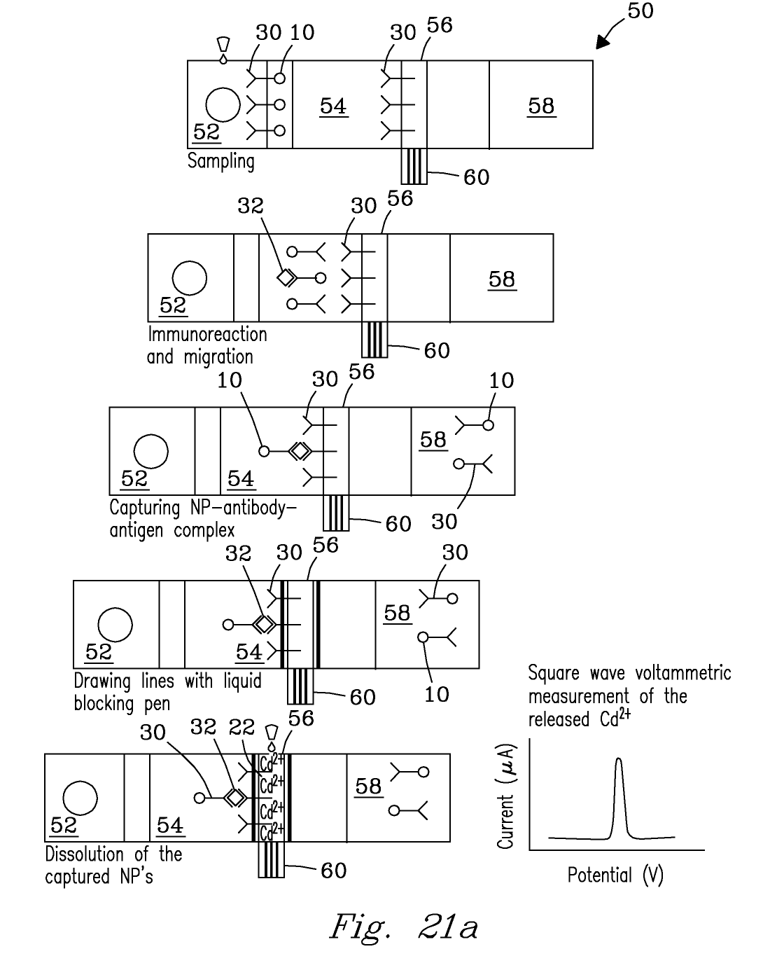

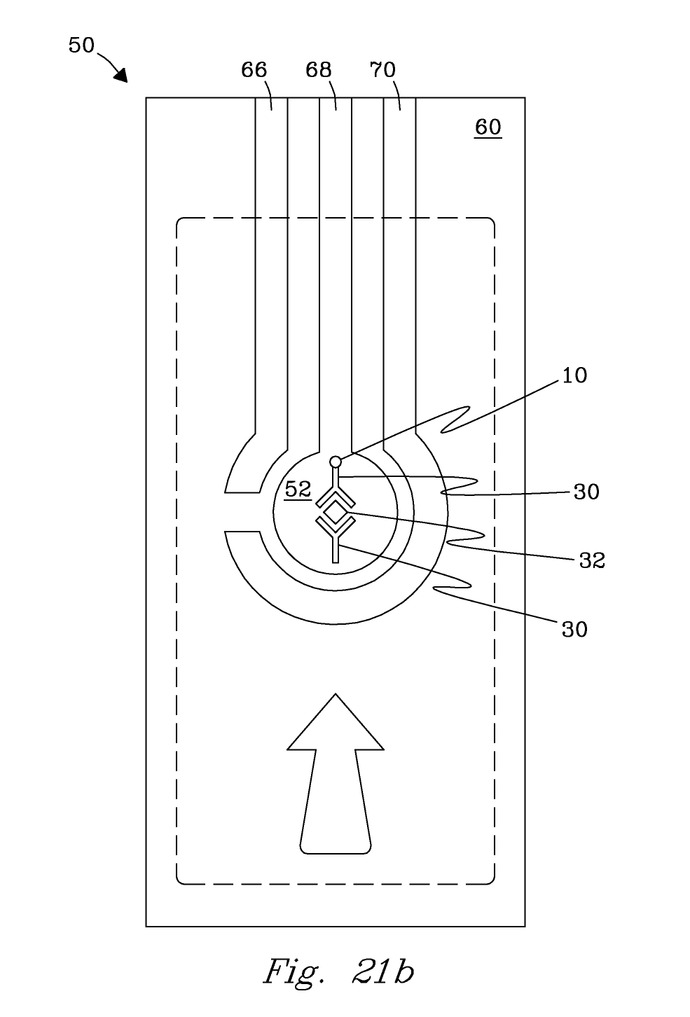

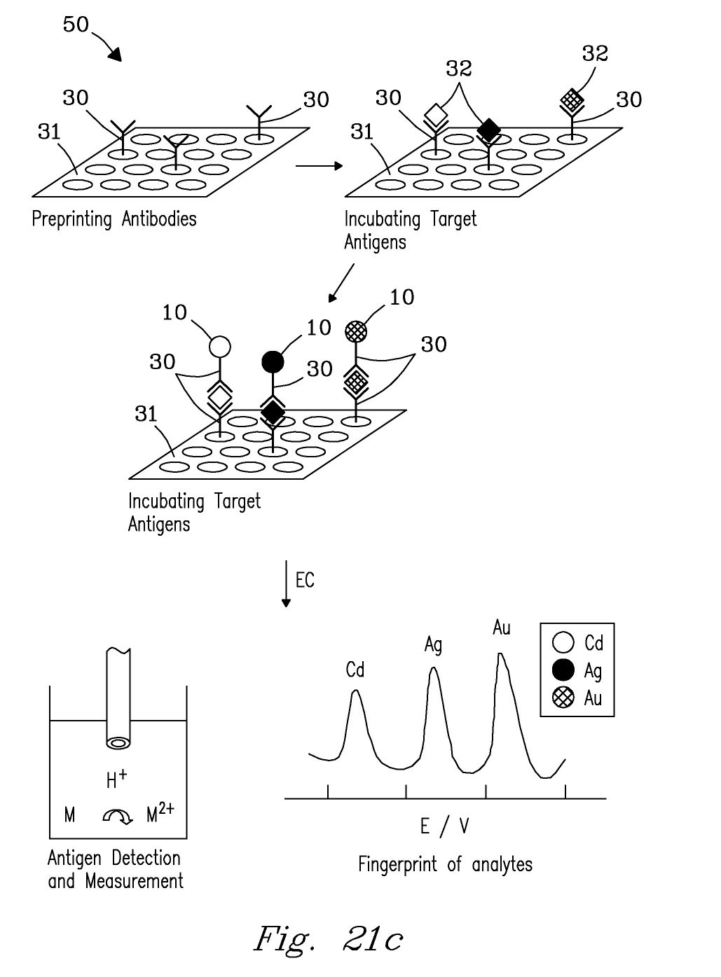

Biosensor platforms that incorporate functionalized apoferritin nanoparticles have been demonstrated. FIGS. 21a-21c illustrate three different biosensor platforms that employ functionalized apoferritin nanoparticles as biomolecular labels. FIG. 21a illustrates components of a first exemplary biosensor that is based on an immunochromatographic/electrochemical platform, according to an embodiment of the invention. In the figure, biosensor 50 is configured as a test strip 50 that includes four zones: sample loading zone 52, contact zone 54, test zone 56, and absorbent (pad) zone 58. Here, the test strip is composed of nitrocellulose, but is not limited thereto. An electrode 60, e.g., a screen printed electrode (SPE, described in reference to FIG. 21b), is coupled to the test zone that provides for measurement of any immunoassay events. Prior to sampling and assay, antibody 30 (primary antibody)—nanoparticle (NP) 10 conjugates are introduced (pre-coated) within contact zone 54. In the instant biosensor, the nanoparticles are encoded with metal phosphate (e.g., cadmium phosphate) as an electrochemical agent, but is not limited thereto. A capture antibody 30 (secondary antibody), is immobilized within test zone 56. At the start of the assay (i.e., sampling), a liquid sample solution (e.g., 100 μL) containing a target antigen (e.g., IgG) 32 is applied to sample loading zone 52. Fluid migrates by capillary action to the opposite end of the strip, and required immunoreactions take place during the fluid migration. As fluid enters contact zone 54 (capturing), antigen 32 in the fluid reacts with the antibody 30 (primary antibody) that is labeled with the apoferritin nanoparticle 10 (i.e., Ab-NP conjugate) to form antigen 32—antibody 30—nanoparticle 10 complexes (i.e., Ag-Ab-NP complexes). The (Ag-Ab-NP) complexes enter test zone 56 where a covalently bound secondary antibody 30 captures antigen 32 in the (Ag-Ab-NP) complexes (using different epitopes) to form sandwich-type nanoparticle 10—antibody 30—antigen 32—antibody 30 complexes (i.e., NP-Ab-Ag-Ab). The (NP-Ab-Ag-Ab) complexes remain in the test e while remaining sample fluid migrates into absorbent zone 58. After a preselected time period (˜minutes), formation of (NP-Ab-Ag-Ab) complexes is completed within test zone 56. Conjugated nanoparticles in the (NP-Ab-Ag-Ab) complexes are dissolved (Dissolution) under acid conditions, and metal ions 22 are released and quantified by electrochemical detection. This biosensor and method are suitable for quantitative analysis of antigen, as electrochemical signals are proportional to the concentration of antigen in the measured samples that complex with the (Ab—NP conjugates) in the immunoassay.

FIG. 21b illustrates components of a second exemplary biosensor 50. The biosensor is a nanoparticle-based immunosensor that detects preselected biomarkers (e.g., proteins, enzymes, antigens, antibodies) found e.g., in blood, saliva, and other biological fluids. The biosensor is disposable following use. The biosensor includes a screen-printed electrode (SPE) 60 that consists of a reference electrode 66, a working electrode 68, and a counter electrode 70. Working electrode 68 serves as a transducer that provides appropriate signal conversion during the assay. SPE 60 includes a sensing area 52 that, in the instant platform, serves as sample zone 52, permitting a sample fluid to be introduced to the biosensor. Prior to immunoassay, (capture) antibody 30 (e.g. anti-IgG) (primary antibody) is immobilized within sample zone 52. When a sample is introduced to the sample zone, antibody 30 (primary antibody) interacts with a target antigen 32 present in the fluid that is further conjugated with a nanoparticle 10 labeled secondary antibody 30 (e.g. anti-IgG) to form a sandwich-type immunocomplex (i.e., Ab-Ag-Ab-NP). Once the assay is completed, functionalized apoferritin nanoparticles 10 are dissolved to release metal ions (not shown) for electrochemical analysis. While an immunoassay event is illustrated, the biosensor is not limited thereto. For example, other events including, e.g., enzyme binding to various chemical adducts are likewise measurable. Thus, no limitations are intended. Biosensor 50 is configured to insert into, e.g., a detection instrument (e.g., a hand-held amperometric or electrochemical reader and display) that provides for measurement of the preselected biomarker in conjunction with the encoded agent.

FIG. 21c illustrates components of a third exemplary biosensor 50. Here, biosensor 50 is a multiplexed immunosensor 50 that is based on a protein microarray platform. Briefly, antibodies 30 specific to three preselected target antigens 32 are preprinted onto microarray slide 31. Slide 31 composition is not limited and can include, e.g., glass or metal (e.g., gold) slides. Type of assay conducted using the microarray platform is not limited. For example, DNA probes can be preprinted, e.g., for DNA assay; or, antibodies may be preprinted, e.g., for protein assay. No limitations are intended. In the instant immunoassay application, target antigens 32 present in a sample when introduced to the microarray are incubated with the preprinted antibodies to form antigen 32—antibody 30 conjugates (Ag-Ab). Secondary antibody 30 labeled with apoferritin nanoparticles 10 (NP) are introduced to microarray 31 and incubated to form sandwich type (NP-Ab-Ag-Ab) immunocomplexes on the microarray slide. In the instant example, apoferritin nanoparticles 10 are encoded with, e.g., three (3) inorganic metal phosphates (e.g., cadmium phosphate, silver phosphate, and gold phosphate) at preselected mole ratios for electrochemical assay. Nanoparticles may be further encoded with, e.g., different optical agents (e.g., dyes) that allow for optical imaging. No limitations are intended. For electrochemical detection, nanoparticles in the sandwich immunocomplexes are dissolved to release encoded metals (here shown as Cd, Ag, and Au) as cations, for measurement of target antigens.

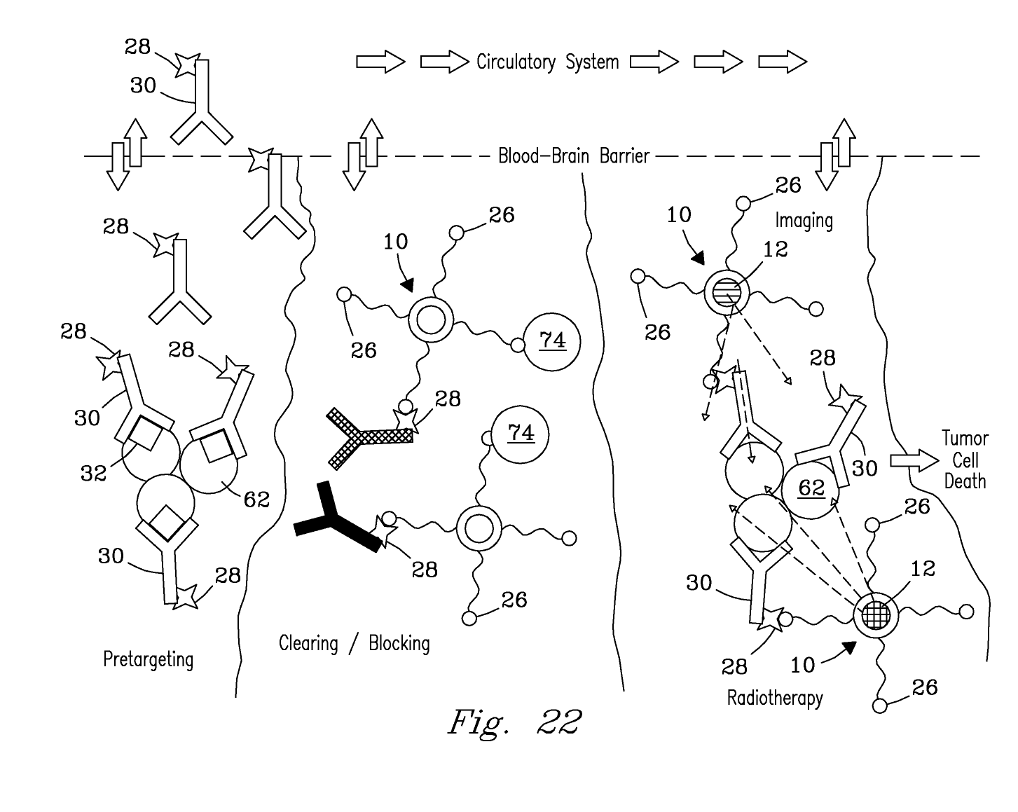

FIG. 22 illustrates an exemplary process for treatment of tumors and cancers that employs functionalized apoferritin nanoparticles, according to an embodiment of the invention. In a pretargeting step, illustrated in the figure, an antibody 30 that targets a specific tumor or cancer antigen 32 (e.g., tumor IgG) is modified with a bridging (linker) protein 28 (e.g., streptavidin) to form streptavidin 28—antibody 30 conjugates (e.g., streptavidin—anti-tumor IgG), which are administered intravenously to a patient. The streptavidin—anti-tumor IgG conjugates penetrate the blood-brain barrier and other membranes and bind to antigens 32 (e.g., tumor IgG antigens) on the surface of tumor cells 62. Following incubation, a clearing-blocking agent is administered that comprises, e.g., a non-encoded nanoparticle 10 modified (functionalized) with biotin 26 further modified with galactose (Gal) 74 sugar that blocks and/or removes (clears) non-specific streptavidin-antibody conjugates (i.e., conjugates that do not target tumor or cancer cells). Following incubation, biotin 26 modified apoferritin nanoparticles 10 encoded with, e.g., a radioimaging agent (e.g. ¹⁵³Gd) are administered to a patient. Once attached, the apoferritin nanoparticle 10 encoded with the a radioimaging agent (radioisotope) can be used for imaging and/or detection of the cancer. Radioimaging agents allow parameters such as organ uptake, target dosing, saturation, radioisotope longevity in the target tumors or cancerous tissues, clearance and metabolism of the radioisotopes, and like parameters to be determined or assessed, which allows parameters for radiotherapy to be predicted. Next, biotin 26 modified apoferritin nanoparticles 10 encoded with, e.g., a radiotherapeutic agent e.g., radium-223 (²²³Ra), a strong gamma emitter, are administered to the patient. The biotin-labeled apoferritin nanoparticles pass through the blood brain barrier and attach to the surface of cancer cells 62 through the biotin 26—streptavidin 28 interaction. The radiotherapeutic agent (e.g., ²²³Ra) with its emission of strong gamma (δ) photons is used to target and kill the cancer or tumor cells. While imaging and radiotherapy have been described in reference to nanoparticles encoded with single radioisotopes, the invention is not limited thereto. For example, as described hereinabove, multiple metals can be encoded within the functionalized nanoparticles, e.g., as metal phosphates. Thus, both imaging and/or radiotherapeutic agents may be encoded within functionalized nanoparticles. Such combinations of encoded agents, e.g., can image a treatment area at the same time that therapeutic agents deliver the necessary radiation doses. In addition, concentrations of each of the encoded agents can be varied. For example, in one application, concentration of the imaging agents may be desired over that of the radiotherapeutic agent or vice versa. All combinations and concentrations of agents as will be selected by those of skill in the art in view of the disclosure are within the scope of the invention. While pretargeting has been described herein with reference to streptavidin-antibody and streptavidin/biotin interactions, interactions are limited thereto. For example, pretargeting methods that employ bispecific antibody—hapten peptide interactions described, e.g., by Sharkey et al. (C A Cancer J Clin 56:226-243; 2006) can be used in conjunction with functionalized apoferritin nanoparticles of the invention. Thus, no limitation in selected modalities and immunocomplex interactions is intended.

Following are examples which will provide an increased understanding of the invention in its many aspects.

The following terms are defined for ease of understanding. Phosphate buffered saline (PBS): A buffer solution typically containing phosphate acids and phosphate (PO₄³⁻) salts (e.g., sodium and/or potassium) and/or optionally other salts (e.g., sodium chloride) used to maintain pH and stability of biomolecular and immunologic complexes in biochemical solutions. TRIS®, also known as trishydroxymethylaminomethane [formula (HOCH₂)₃CNH₂] (C₄H₁₁NO₃) [CAS No. 77-86-1] is a primary amine used to maintain pH in buffered solutions. TWEEN-20®, also known as polyoxyethylene (20) sorbitan monolaurate [CAS No. 9005-64-5] (C₅₈H₁₁₄O₂₆), is a polysorbate nonionic surfactant used as a blocking agent in biochemical applications. TRIS®-buffered saline (TBS): A buffer solution containing. Blocking Buffers: A buffer solution (e.g., PBS and TBS) containing at least one blocking agent (e.g., 1% Bovine Serum Albumin or BSA) that binds to nonspecific target sites in biochemical and immunologic complexes, e.g., protein/antibody, antibody/antigen and the like. Blocking buffers minimize background without altering the desired binding interactions thereby maximizing sensitivity and signal-to-noise (S/N) in assays and immunoassays. Typical blocking buffers include, but are not limited to, e.g., BSA Blocking Buffers, e.g., BSA in PBS (i.e., PBSB buffer); and BSA in TBS. PBST buffer: a blocking buffer containing phosphate buffered saline (PBS) and TWEEN-20® (e.g., PBS containing 0.5% TWEEN-20®). TRIS®-HCl Buffer: A buffer solution containing TRIS® and hydrochloric acid (HCl) that provides pH buffering of a solution in the range from about 7.5 to about 9.0). TT or TTL buffer: A buffer containing TRIS®-HCl and TWEEN-20® (e.g., 250 mM TRIS®-HCl, pH 8.0; and 0.1% TWEEN-20®). Hybridization buffer: A buffer solution containing various salts (e.g., NaCl and sodium citrate (e.g., 750 mm NaCl, 150 mm sodium citrate) used as a diluent for oligonucleotide probes involved in biochemical hybridization reactions, e.g., as described herein. Bicinchoninic Acid Assay (BCA Assay): A calorimetric, biochemical assay, for determining concentration of protein in a solution, e.g., as described by Smith et al. (“Measurement of protein using bicinchoninic acid”, Anal. Biochem. 150: 76-85 (1985). Total protein concentration is determined as a function of color change exhibited in sample solutions in proportion to protein concentration, which can then be measured using calorimetric techniques.

### EXAMPLE 1

### Preparation of Metal Phosphate Encoded Apoferritin Nanoparticles

### Encapsulation Method

Cadmium phosphate encoded apoferritin nanoparticles were prepared as follows. Apoferritin was first diluted with dilute (˜0.01 M) phosphate buffered saline (PBS) and loaded on a desalting column (e.g., a PD-10 desalting column) packed with a cross-linked dextran gel (available under the tradename SEPHADEX-25®), and washed with PBS buffer to obtain purified apoferritin. Purified apoferritin solution was adjusted to pH 2 with 1M HCl while magnetically stirring. Cadmium chloride (10 mM) (alternatively, lead nitrate, zinc nitrate, or other metal nitrate, including mixtures of metals at different concentrations or ratios) was slowly added to the apoferritin solution. pH was adjusted to pH 8.5 with dilute (0.1 M) NaOH added dropwise. Mixture was stirred continuously to form a metal phosphate core inside the apoferritin cavity. Mixture was centrifuged and washed with (0.1 M) TRIS®-HCl buffer using a filter having a molecular weight cutoff (MWCO) value of 25000. Nanoparticles were reassembled in solution to form metal phosphate encoded apoferritin nanoparticles. Protein concentration was determined using a bicinchoninic acid (BCA) assay. Metal concentrations were determined by ICP/AES.

### EXAMPLE 2

### Preparation of Metal Phosphate Encoded Apoferritin Nanoparticles

### Diffusion Method

Cadmium chloride (10 mM) (alternatively, lead nitrate, zinc nitrate, or other metal nitrate, including mixtures of metals at different concentrations or ratios) was slowly added to purified apoferritin solution (prepared in 0.1 M TRIS® buffer, pH=8.0). Mixture was stirred continuously to diffuse cadmium ions into the apoferritin core. Dilute phosphate buffer (0.2 M, pH=7.0) was introduced dropwise into the solution to form metal phosphate within the apoferritin core. Excess metal cations outside apoferritin nanoparticles were precipitated with phosphate buffer and centrifuged. Supernatant was washed with (0.1 M) TRIS®-HCl buffer using a filter with a MWCO of 25000. Apoferritin nanoparticles were reassembled in solution to form metal phosphate encoded apoferritin nanoparticles.

### EXAMPLE 3

### Preparation of Marker Encoded Apoferritin Nanoparticles

### Encoded with: Fluorescence and Redox Markers

In a first case, a fluorescence marker (fluorescein, as a sodium salt) was used to encode apoferritin nanoparticles for use in a fluorescence microscope immunoassay. Apoferritin solution (equine spleen) was prepurified on a gel-filtration column to remove aggregates. Eluent fractions (0.1 M ammonium acetate, pH 7.0) were collected, mixed, and concentrated using a centrifugal filter and washed with autoclaved water using the same filter. Purified apoferritin solution (1.1×10⁻⁵ M), was gradually adjusted and maintained at pH 2 by slow addition of dilute HCl solution. Fluorescein solution was slowly added and pH was slowly raised to 8.5 by addition of dilute NaOH solution. Resulting solution was stirred and concentrated using a centrifugal filter device and washed with autoclaved water using the same filter. Solution was exhaustively dialyzed with dilute 0.05 M phosphate buffer (pH 7.4) using a spectra/Por float-A-lyzer with a molecular weight cutoff (MWCO) of 25000 Da to remove free fluorescein. Fluorescein encoded apoferritin nanoparticles were purified on a desalting column with exclusion limit 5000 using a dilute phosphate buffer as eluent (pH 7.4). Collected fractions were mixed together and concentrated. For control experiments, fluorescein was added to an apoferritin solution at the same levels. pH was varied only between 4.0 and 5.0 to prevent apoferritin from disassembling into subunits. In a second case, a redox marker (hexacyanoferrate as a potassium salt) was used to encode apoferritin nanoparticles for use in an electrochemical immunoassay. Here, a 0.5 M K₃Fe(CN)₆ solution was used and final concentration of hexacyanoferric acid in the mixture was 0.1 M.

### EXAMPLE 4

### Functionalization of Marker Encoded Apoferritin Nanoparticles

### Encoded with: Fluorescence and Redox Markers

### Functionalized with: Biotin

Apoferritin nanoparticles encoded with fluorescein or hexacyanoferrate as markers were functionalized with biotin as follows. Suspensions containing encoded apoferritin nanoparticles were mixed at room temperature with Biotin-NHS coupling reagent (i.e., biotinamidohexanoyl-6-amino-hexanoic acid N-hydroxy-succinimide ester), prepared in dilute (0.05 M) phosphate buffer. After incubation, mixture was exhaustively dialyzed with dilute phosphate buffer using a spectra/Por float-A-lyzer with a molecular weight cutoff (MWCO) of 25000 Da to remove any free Biotin-NHS. Biotin-functionalized nanoparticles were concentrated, mixed with PBSB buffer containing phosphate buffer (PBS) (pH 7.4) and 0.1% BSA), and stored at 4° C.

### EXAMPLE 5

### Fluorescence Immunoassay

Example 5 presents an exemplary protocol for conducting immunoassay that employs apoferritin nanoparticles encoded with preselected fluorescence markers (agents). An aldehyde-modified glass slide was washed with autoclaved water and nitrogen dried. Slide was spotted with 0.2 μL (per spot) of antibodies (anti-IgG, 1.0 mg/mL) and incubated overnight in a sealed Petri dish saturated with water vapor. Each spot area was marked with marker pen on the opposite side of the slide, and the antibody-spotted slide was washed extensively with phosphate buffer (0.05 M phosphate buffer containing 0.1% w/w SDS, pH 7.4). The slide was blocked (i.e., nonspecific binding sites were blocked) with PBSB buffer containing 1% BSA in dilute phosphate buffered saline (PBS), followed by treatment with 60 mM sodium borohydride solution containing 25% ethanol to minimize nonspecific binding. The slide was then exposed to antigen (e.g., mouse IgG) solution by dropping (˜10 μL) a desired concentration of antigen into each spot area. Immunoreaction was allowed to proceed in a sealed Petri dish saturated with water vapor. Slide was then washed with PBSB buffer solution. The coated spot containing the antibody-antigen complex was exposed to a biotin-modified secondary antibody (10 μL for each spot, 1 mg/mL), incubated, and washed. Streptavidin solution (e.g., 10 μL of 1 mg/mL) was then added to each spot and the biotin-streptavidin interaction was allowed to proceed (˜30 min). Following washing, a solution containing biotin-modified marker (e.g., fluorescein) encoded apoferritin nanoparticles was added to each spot and the reaction was allowed to proceed (˜30 min). After washing with PBSB, fluorescence microscope images were taken, e.g., using an inverted optical microscope integrated with CCD camera.

### EXAMPLE 6

### Electrochemical Immunoassay

Example 6 presents an exemplary protocol for conducting electrochemical immunoassays that employs apoferritin nanoparticles encoded with preselected electrochemical agents. Generalized electrochemical immunoassay is described, e.g., in a Bangs Laboratory procedure [Technote 101, 2002, Bangs Laboratories Inc., Fishers, Ind.]. Here, electrochemical immunoassays were modified to incorporate use of biotin functionalized, hexacyanoferrate encoded apoferritin nanoparticles of the invention for electrochemical detection. Briefly, 50 μL of magnetic beads (microspheres) coated with antibody (e.g., anti-mouse IgG) suspended in PBSB buffer were mixed with 10 μL of a preselected concentration of antigen (e.g., IgG). The immunoreaction was allowed to proceed (˜60 min) under shaking conditions. Resulting antibody-antigen coated microspheres were washed with PBSB buffer and resuspended in PBSB. 10 μL of biotin-modified secondary antibodies were added and incubated under shaking conditions, followed by magnetic separation and washing with PBSB buffer. Magnetic beads were resuspended in PBSB buffer, streptavidn was added, and the streptavin-biotin interaction was allowed to proceed (˜30 min), followed by magnetic separation and washing. Beads were resuspended in PBSB buffer, and biotin-functionalized apoferritin nanoparticles encoded with hexacyanoferrate (redox marker) were added. Following incubation, magnetic separation, and washing, HCl—KCl solution (˜50 μL, 0.1 M) was added to release hexacyanoferrate from the encoded apoferritin nanoparticles. The solution containing released hexacyanoferrate was transferred to a screen-printed electrode (SPE) connected to an electrochemical analyzer via a sensor connector for square wave voltammetric (e.g., SWV) measurement. The SPE electrode consisted of a carbon working electrode, carbon counter electrode, and Ag/AgCl reference electrode. After cleaning the electrode surface with dilute (0.05 M) phosphate buffer (pH 7.4) at a 1.5 V potential and drying with air, a droplet of sample solution (˜50 μL) was placed in the area of the three electrodes. Potential was scanned from 0 V to 0.45 V a step of 4 mV, amplitude 25 mV.

### EXAMPLE 7

### Functionalization of Metal Phosphate Encoded Apoferritin Nanoparticles

Example 7 presents an exemplary protocol for functionalization of encoded apoferritin nanoparticles encoded with preselected electrochemical agents that find use in electrochemical immunoassays. Metal phosphate encoded apoferritin nanoparticles were prepared as described herein. Apoferritin solution was prepurified on a desalting column (e.g., a PD-10 desalting column) to remove aggregates. Collected eluent fractions (0.1M ammonium acetate, pH 7.0) were mixed and concentrated with a centrifugal filter device and washed with autoclaved water using the same filter. Autoclaved water was then added. Cadmium nitrate (10 mM solution) (or lead nitrate and/or other metal nitrate) was added slowly into the purified apoferritin solution at pH 8.0 and the mixture was continuously stirred to allow cadmium ions to diffuse into the apoferritin cavity (core). Subsequently, dilute (0.2M) phosphate buffer (pH 7.0) was slowly introduced to form the metal phosphate core. Excess metal cations outside the apoferritin core were precipitated with phosphate buffer and separated by centrifugation. Supernatant was passed through a filter with a molecular weight cutoff (MWCO) of 25000 and the recovered apoferritin nanoparticles were washed with 0.1M TRIS®-HCl buffer solution using the same filter. Apoferritin nanoparticles were reassembled in TRIS®-HCl solution to form metal phosphate encoded apoferritin nanoparticles. Protein concentration was determined using a BCA assay with bovine serum albumin (BSA) used as a standard.

Encoded apoferritin nanoparticles and antibody-modified metal phosphate encoded apoferritin nanoparticles were functionalized with biotin by mixing suspensions of encoded apoferritin nanoparticles with biotin-NHS reagent (prepared in dilute (0.05M) phosphate buffer, pH 7.4) at room temperature. After incubation, mixtures were extensively washed with dilute phosphate buffer to remove any free biotin-NHS using a filter with molecular weight cutoff (MWCO) of 25000. Resulting functionalized nanoparticles were concentrated, after which dilute phosphate buffer (pH 7.4) containing 0.1% BSA was added (˜0.4 mL) and stored at 4° C. Biotin-modified, lead phosphate encoded, apoferritin nanoparticles were prepared similarly. Antibodies (e.g., anti-TNF-α and anti-MCP-1) were conjugated with cadmium phosphate encoded, and lead phosphate encoded, apoferritin nanoparticles, respectively, using 3-(3-dimethylaminopropyl)-1-ethylcarbodiimide (EDC) and NHS coupling reagents, respectively.

### EXAMPLE 8

### Preparation of Functionalized Apoferritin Nanoparticles Encoded with Surrogate Radioisotopes

Lutetium phosphate encoded apoferritin nanoparticles were prepared by the diffusion method described in Example 2. Apoferritin solution was purified using 0.1 M TRIS®-HCl buffer as eluent. Collected fractions were concentrated and incubated (˜1 hr) with desired concentrations of lutetium chloride (e.g., 1, 3, 5, 10 mM) to diffuse lutetium into the apoferritin cavity. Dilute (˜0.2 M) phosphate buffer (pH 7.0) was slowly introduced and the mixture was stirred to form lutetium phosphate in the apoferritin cavity (core). Excess metal cations outside apoferritin were precipitated by addition of phosphate buffer, and separated by centrifugation. Supernatant was passed through a PD-10 desalting column to remove excess small molecule components with dilute (˜0.01 M) phosphate buffer as eluent. Concentrated lutetium-phosphate encoded apoferritin nanoparticles were reassembled and subjected to bicinchoninic acid (BCA) assay and ICP analysis to determine protein concentration and core lutetium concentration, respectively. XPS analysis confirmed that lutetium phosphate was located within the apoferritin core. Approximately 500 lutetium atoms loaded into each apoferritin nanoparticle using 10 mM lutetium chloride as the precursor. Saturation was achieved at ˜5 mM lutetium chloride. Precursor concentration higher than 10 mM led to protein aggregation. Replicate samples (e.g., 6) at each lutetium chloride concentration gave a relative standard deviation of less than 10%. Nanoparticles were subsequently functionalized with biotin using biotin-NHS reagent. Biotin-functionalized yttrium phosphate apoferritin nanoparticles were prepared similarly.

### EXAMPLE 9

### Functionalized Apoferritin Nanoparticles Encoded with Radioisotope Surrogates as a test for Encoded Radioisotopes Suitable for RadioImmunoassay, Radioimmunoimaging, and Radioimmunotherapy

Pre-targeting capability of biotin-modified lutetium phosphate encoded apoferritin nanoparticles conjugated with tags comprising FITC-streptavidin and avidin-modified magnetic beads was tested. Streptavidin-modified magnetic beads (˜5 μL) were mixed (˜1 min) with (˜100 μL) (PBSB) buffer (phosphate buffered saline containing 1% BSA) to block active sites of the magnetic beads (i.e., to minimize non-specific binding). After magnetic separation and washing with PBST buffer [phosphate buffer (PBS) containing 0.5% TWEEN-20®], the beads were suspended in PBS buffer solution (e.g., 40 μL), and solution containing biotin-functionalized lutetium phosphate encoded apoferritin nanoparticles was added (10 μL) and incubated (˜30 min) at room temperature. After magnetic separation, biotin-functionalized, lutetium phosphate encoded apoferritin nanoparticles attached to magnetic beads were washed with PBST buffer and suspended in PBS buffer. FITC-streptavidin (˜5 μL 1 ppm) was added, mixed, and incubated (˜30 min). Following separation and washing, magnetic beads bearing the FITC/lutetium phosphate encoded apoferritin nanoparticle complex were resuspended in PBS buffer. Complexes were measured at 460 nm excitation using fluorescence spectroscopy.

### EXAMPLE 10

### Functionalization of Encoded Apoferritin Nanoparticles with DNA Probes for Quantitative Electrochemical Assay of DNA

Apoferritin nanoparticles encoded with hexacyanoferrate were functionalized (FIG. 15) by attaching an amino modified DNA probe using a coupling reagent, EDC, as described hereafter. Generalized coupling of DNA probes is described, e.g., in a Bangs Laboratories procedure [Product Data Sheet 644, Bangs Laboratories Inc., Fishers, Ind.]. A suspension containing hexacyanoferrate encoded apoferritin nanoparticles was mixed with an (˜1000 ppm) amino-modified DNA probe (e.g., Probe 1) having, e.g., oligonucleotide sequence: [5′-ACA CTG GGG GGG CTA GGG AA-3 amino] in freshly prepared coupling buffer (100 mM EDC, 100 mM imidazole buffer, pH 7.0), and incubated at 50° C. under continuous rotation or inversion (˜for 3 h). Mixture was washed using a filter with an MWCO of 10000 to remove free DNA probe and EDC. Solution was concentrated and phosphate buffer (0.4 mL, 0.05 m, pH 7.4) containing 0.1% BSA was added. Solution was stored at 4° C.

### EXAMPLE 11

### Bioassay Applications of Encoded Apoferritin Nanoparticles Functionalized with DNA-Probes for Electrochemical Detection of DNA

DNA hybridization experiments were performed using a modified Bangs Laboratories procedure [Technote 101, 2002, Bangs Laboratories Inc., Fishers, Ind.], modified with use of biotin functionalized, hexacyanoferrate encoded apoferritin nanoparticles of the invention as labels for electrochemical detection. Hexacyanoferrate encoded apoferritin nanoparticles were functionalized with a first DNA probe (e.g., Probe 1, EXAMPLE 10). Streptavidin-coated magnetic beads (5 mL, 10 mg/mL) were washed with TTL buffer (95 mL, 100 mm Tris-HCl, pH 8.0, 0.1% TWEEN-20®, and 1M LiCl) and suspended in TTL buffer (21 mL). A biotinylated DNA probe (e.g., Probe 2) having, e.g., oligonucleotide sequence: [5′-biotin-CAA AAC GTA TTT TGT ACA AT-3′] (4 mL, 1000 mg/L) was added, and the mixture was incubated under shaking conditions (˜30 min). Probe-coated magnetic beads were washed with TT buffer (95 mL, 250 mM Tris-HCl, pH 8.0; 0.1% TWEEN-20®) and suspended in PBSB buffer (50 mL, 0.05 m phosphate buffer (pH 7.4), 1% BSA). Following magnetic separation, surfaces of DNA probe-coated magnetic beads were blocked with PBSB buffer (˜30 min) and dispersed in hybridization buffer (750 mm NaCl, 150 mm sodium citrate). Desired concentration of a target DNA having, e.g., oligonucleotide sequence: [5′-TTC CCT AGC CCC CCC AGT GTG CAA GGG CAG TGA AGA CTT GAT TGT ACA AAA TAC GTT TTG-3′] was added, and the mixture was incubated under shaking conditions (˜60 min). Resulting hybrid-conjugated microspheres (beads) were washed with TT (TTL) buffer and suspended in hybridization buffer, and followed by addition of DNA probe 2—functionalized apoferritin nanoparticles (10 mL). Mixture was incubated (˜60 min.), magnetically separated, and washed with TT buffer. 50 mL of 0.1M HCL/KCL was then added to release hexacyanoferrate from the encoded apoferritin nanoparticles for electrochemical measurement. The HCl/KCl solution containing released hexacyanoferrate was transferred to a screen-printed electrode for measurement, as described in Example 6, scanned at a potential from 0 to 0.6 V with a step of 4 mV and an amplitude 25 mV.

### EXAMPLE 12

### Functionalization of Encoded Apoferritin Nanoparticles with a Nucleotide

Guanine-modified metal phosphate (e.g., cadmium phosphate) encoded apoferritin nanoparticles were prepared by attaching a monobase, guanosine 5′-monophosphate, to the nanoparticles through their 5′ phosphate group via the formation of a phosphoramidite bond with the free amino groups of the apoferritin nanoparticle. Guanosine 5′-monophosphate solutions were prepared using TBS buffer solution [(20 mM TRIS®-HCl buffer containing 20 mM NaCl (pH 7.0)]. Subsequently, a guanosine 5′-monophosphate solution at a preselected concentration was mixed with a metal phosphate encoded nanoparticle suspension, and the mixture was shaken (˜1 hour), followed by separation in a desalting column (e.g., a PD-10 desalting column) packed with a cross-linked dextran gel (available under the tradename SEPHADEX-25®). Eluent fractions were concentrated with a centrifugal filter and washed with TBS buffer using the same filter. Purified guanine-modified metal phosphate encoded nanoparticle conjugates were dispersed in TBS to accomplish base-pairing without further alterations.

### EXAMPLE 13

### Nucleotide Functionalized Metal Phosphate Encoded Apoferritin Nanoparticles for Quantitative Electrochemical Detection of Single Nucleotide DNA Polymorphisms

Electrochemical quantification of single-nucleotide polymorphisms (SNPs) was performed in concert with nucleotide functionalized metal phosphate encoded apoferritin nanoparticles, described hereafter.

(Step 1): DNA Hybridization. In a first case, sequential DNA hybridization reactions were followed (see FIG. 18). Biotinylated DNA probes (25 μL, 1 nmol) and a desired concentration of a mismatched (mutant) DNA having, e.g., oligonucleotide sequence [5′-ACT GCT AGA CAT TTT CCA CAT-3′] (i.e., mutated at a cytosine site, illustrated as “C” in FIG. 18) was mixed in a centrifuge tube, and incubated under gentle mixing (˜1 hour). Complementary DNA (25 μL, 2 nmol.) having, e.g., oligonucleotide sequence [5′-ACT GCT AGA GAT TTT CCA CAT-3′] was added, and the hybridization reaction was allowed to proceed (˜1 hour).

In a second case, one-step DNA hybridization reactions were followed (see FIG. 19). Biotinylated DNA probes (25 μL, 1 nmol), a desired concentration of a mismatched (mutant) DNA having, e.g., oligonucleotide sequence [5′-ACT GCT AGA CAT TTT CCA CAT-3′] (i.e., mutated at a cytosine site, illustrated with “C” in FIG. 19), and complementary DNA (25 μL, 2 nmol.) having, e.g., oligonucleotide sequence [5′-ACT GCT AGA GAT TTT CCA CAT-3′] were mixed in a centrifuge tube, and incubated under gentle mixing (˜90 min). Electrochemical response of cytosine mutant target DNA (˜50 μM) showed electrochemical signal increased as a function of hybridization time, indicating an increase in amount of cytosine mutant sites on the duplexed DNA and leading to an increase in quantity of coupled cadmium phosphate encoded apoferritin nanoparticle probes. Here, response signals were stable after 90 min, which used as the hybridization reaction time.

(Step 2). Magnetic Capturing of any Duplexed DNA. Magnetic capturing of duplexed DNA was carried out using streptavidin-modified magnetic beads (see FIG. 18 and FIG. 19). Streptavidin-coated magnetic beads (˜5 μL) were washed with (˜95 μL) TTL buffer (100 mM TRIS®-HCl, pH 8.0, 0.1% Tween, and 1 M LiCl). After magnetic separation, the suspension was removed. Beads were resuspended above the DNA mixture (from Step 1) containing the formed duplex DNA and the excess of complementary DNA. The mixture was incubated for 30 min with gentle mixing. The magnetic beads, coated with the formed duplex DNA, were washed twice with 95 μL of TT buffer (250 mM Tris-HCl, 0.1% TWEEN-20®) and blocked for 15 min with 100 μL of TT buffer containing 1% bovine serum albumin (BSA). The beads were washed twice with 95 μL of TT buffer and resuspended in 45 μL of 20 mM TBS (pH 7.8) with 60 mM KCl and 10 mM MgCl2.

(Step 3). Hybridization between Mismatched Sites of Duplexed DNA and Guanine-modified metal phosphate encoded nanoparticles. Guanine-modified (G-modified) metal phosphate (e.g., cadmium phosphate) encoded apoferritin nanoparticles (5 μL), prepared as described in EXAMPLE 12, were added to duplexed DNA-coated magnetic beads in solution in the presence of (“Klenow” fragment) DNA polymerase I (0.5 U/μL), and mixed at room temperature (˜for 1 hour). After incubation, the magnetic-bead/DNA/G-modified metal phosphate nanoparticle complexes were washed with TT buffer (95 μL) to remove any nonspecifically bound G-modified, metal encoded nanoparticle conjugates and resuspended in (˜50 μL) 0.2 M acetate buffer (pH 4.6) containing mercury(ii) atomic absorption standard solution (10 μg/mL). Cadmium ions were released from the apoferritin cadmium phosphate core in the acetate buffer at pH 4.6. After mixing and magnetic separation, the acetate buffer containing dissolved cadmium ions was transferred to a screen-printed electrode (SPE) for electrochemical analysis.

(Step 4). Electrochemical Detection. Dissolved cadmium ions were measured with square wave voltammetry (SWV) using an in situ plated mercury film on the SPE with a 1 min pretreatment at +0.6 V, followed by a 2 min accumulation at −0.9 V. After a 15 sec. rest period (without stirring), stripping was performed by scanning the potential from −0.9 to −0.5 V, with a step potential of 4 mV, an amplitude of 25 mV, and a frequency of 25 Hz.

### EXAMPLE 14

### Determination of SNP Frequencies in Constructed DNA Samples

Quantification of SNPs is important, e.g., to estimate SNP frequency in DNA sample pools. To demonstrate ability to quantify SNP frequencies, cytosine-mutated DNA targets (as mutant SNP alleles) and perfect-matched DNA (as wide-type SNP alleles) were used to construct an artificial DNA pool. Mutant DNA and perfect-matched DNA were mixed at different ratios ranging from 0 to 100% for use as constructed DNA samples. Biotinylated DNA probes (25 μL, 1 nmol) were mixed with each of the constructed DNA samples (50 μL). Electrochemical measurements of the constructed DNA samples were obtained by following the one-step hybridization procedure, described in EXAMPLE 13 (Steps 1 through 4). SNP frequency was then calculated using equation [1]:

$$S N P Frequency = ( \frac{I}{{I}_{o} + {I}_{100}} ) \qquad [ 1 ]$$

Here, (I) is the current intensity produced by the constructed DNA pool sample (containing mutant DNA and perfect-matched DNA), (I₀) is the current intensity produced by the perfect-matched DNA sample (without mutant DNA), and (I₁₀₀) is the current intensity produced by the mutant DNA sample (without perfect-matched DNA). Samples containing perfect-matched DNA, mutant DNA, and an equal molar mixture of perfect-matched DNA and mutant DNA were analyzed. Negligible signals were obtained in samples containing perfectly-matched DNA (0% mutant DNA). As expected, signals for equimolar (1:1) mixtures of perfectly matched DNA and mutant DNA were smaller than those of (100%) mutant DNA samples. Results were reproducible and reliable, indicating the method is applicable for SNP frequency analysis.

### CONCLUSIONS

Apoferritin can be used as a template to prepare single-component and multiple component metal nanoparticles, each with distinct voltammetric signatures. Encapsulation and diffusion approaches have been demonstrated. Encapsulation enables the successful control of the multiple metal composition ratios in compositionally encoded nanoparticles. The new templated synthesis of metal phosphate nanoparticles is simple and fast. The resulting electrochemical signatures from the compositionally encoded nanoparticle tags correlate well with predetermined concentration ratio and indicate a reproducible encapsulation process. The new encoded metallic phosphate nanoparticles thus represent a useful addition to the particle-based product-tracking/identification/protection. The encoded nanoparticles also offer great promise for multiplex electrochemical biosensors and bioassays.

A versatile bioassay label has been disclosed that is based on an apoferritin templated nanoparticle loaded with specific markers that are applicable for biosensing applications, e.g., for sensitive protein detection. Disassembly and reassembly characteristics of apoferritin as a function of pH, as well as the cavity structure of apoferritin provide a facile route to prepare functionalized apoferritin nanoparticles. Optical, electrochemical, and other properties of prepared nanoparticles are easily controlled by loading different and preselected markers and constituents into the apoferritin cavity. While embodiments of the invention have been described and demonstrated in the context of use of a fluorescence marker (fluorescein anion) and a redox marker (hexacyanoferrate anion) in fluorescence microscope immunoassay and electrochemical immunoassay, respectively, the invention is not limited thereto. It will be apparent to those skilled in the art that many changes and modifications may be made without departing from the invention in its true scope and broader aspects. For example, processes described herein could be readily extended to other markers, or to load various contrasting agents and imaging agents and radiotherapy agents and heterogeneous metals for multiplex immunoassays, or to deliver drugs and cell imaging compounds to specific and/or target tissues and cells within a host or patient. In addition, in other applications, simultaneously loading multiple markers into the apoferritin nanoparticles is also possible and may be used, e.g., as a means to build a molecular library of various markers. Various redox and optical makers can be loaded into the cavity of apoferritin nanoparticles in order to develop different nanoparticle labels for optical and electrochemical bioassays. For example, methods disclosed herein have potential to permit capture of molecules including drugs, e.g., for release in various therapeutic applications. The new nanoparticles described herein have also been demonstrated to be suitable as biochemical labels for applications that include bioassays, in particular, immunoassays. They may also be applicable to various other biological assays and immunoassays, including protein and DNA assays. Thus, no limitations are intended by the markers described herein.

A simple, fast, and efficient method has also been disclosed to synthesize apoferritin nanoparticles encoded with radioisotopes, which has been demonstrated using radioisotope surrogates of both lutetium and yttrium phosphates. Radioisotope encoded apoferritin nanoparticles should exhibit both sufficient loading and chemical stability. As such, apoferritin-based synthesis may have high potential for applications in both diagnostics and therapy of cancers. Amino acids present at the channels ends of the apoferritin core, with its many was easily functionalized with biotin before and after the loading, which can be used as radioactive labels in pretargeting technique. With the pretargeting technique, the biotinylated apoferritin loaded with radioactive yttrium nanoparticles will target avidin-conjugated antibody bounded to specific tumor cells. Therefore, the treatment of tumor cells can be realized with the suitable probes. Apoferritin-templated yttrium phosphate nanoparticles offer great promise for radioimmunotherapy of various types of cancers. For example, lutetium-177 (¹⁷⁷Lu) can be loaded within the apoferritin cavity (core), as described herein for non-radioactive surrogates, in a stable phosphate form. Lutetium-177 emits low-energy beta radiation and gamma radiation, which, with its long half-life, should be suitable for both radioimmunotherapy and radioimmunodetection. This apoferritin templated approach significantly improves loading capacity and stability in biological environments. Here, apoferritin is easily functionalized, e.g., with biotin or other functional groups or molecules after the encoding (loading) of the isotope. The functionalized radioisotope encoded nanoparticle can then be used, e.g., as a radioactive label using a pre-targeting technique in which biotinylated apoferritin loaded with radioisotope encoded nanoparticle targets, e.g., an avidin-conjugated antibody bound that binds to specific tumor cells. These radioisotope encoded apoferritin nanoparticles can have potential to be used for diagnosis and radiotherapy treatment of tumor cells, and for radioimmunotherapy and radioimmunodetection of various cancers.

An electrochemical method based on use of nanoparticle probes for quantification of single-nucleotide polymorphisms (SNP). This new SNP detection technology is based on DNA polymerase I-induced coupling of nucleotide-modified nanoparticles (probes) to mutant sites of duplex DNA under the Watson-Crick base-pairing rule. As demonstrated herein, electrochemical analysis is effective at measuring metal released from metal phosphate encoded nanoparticles for quantitative analysis of nucleic acid without, e.g., preamplification. The approaches are expected to provide accurate, sensitive, rapid, and low-cost detection of SNPs.

The appended claims are intended to cover all such changes and modifications as fall within the spirit and scope of the invention.

1. A functionalized apoferritin nanoparticle, comprising:
an apoferritin molecule having a functionalized outer surface that surrounds a preselected agent.

2. The nanoparticle of claim 1, wherein said functionalized outer surface includes at least one surface member selected from the group consisting of: a protein; an antibody; an antigen; a nucleotide; a nucleic acid; a hapten; an aptamer; and combinations thereof.

3. The nanoparticle of claim 1, wherein said functionalized outer surface includes two or more members selected from the group consisting of: a protein; biotin; an antibody; an antigen; a nucleotide; a nucleic acid; a hapten; an aptamer; and combinations thereof.

4. The nanoparticle of claim 3, wherein said two or more members include at least two preselected antibodies that each bind with a preselected target antigen different from the other.

5. The nanoparticle of claim 1, wherein said functionalized outer surface includes at least one member selected from the group consisting of: a protein; biotin; avidin; streptavidin; an antibody; a nucleotide; a nucleic acid; a hapten; an aptamer; and combinations thereof; and said preselected agent includes at least two members selected from the group consisting of: a metal; a metal containing agent; a therapeutic agent; radiotherapeutic agent; an oncology agent; a radioisotope; a magnetic agent; a contrast agent; an imaging agent; an optically-active agent; a calorimetric agent; a fluorescence agent; an electroactive agent; an electrochemical agent; a redox agent; and combinations thereof.

6. The nanoparticle of claim 1, wherein said preselected agent is selected from the group consisting of: a metal; a metal containing agent; a therapeutic agent; an oncology agent; a radioisotope; a radiotherapeutic agent; a magnetic agent; a contrast agent; an imaging agent; an optically-active agent; a colorimetric agent; a fluorescence agent; an electroactive agent; an electrochemical agent; a redox agent; and combinations thereof.

7. The nanoparticle of claim 6, wherein said imaging agent includes a member selected from the group consisting of: gamma camera imaging agents; and position emission imaging agents.

8. The nanoparticle of claim 7, wherein said gamma camera imaging agents include a radioisotope that emits gamma energies in the range between about 80 and 450 keV selected from the group consisting of: copper-67 (⁶⁷Cu); lutetium-177 (¹⁷⁷Lu); rhenium-186 (¹¹⁶Rh); rhenium-188 (¹⁸⁸Rh); technetium-99m (⁹⁹mTc); indium-111 (¹¹¹In); gadolinium-153 (¹⁵³Gd); and combinations thereof.

9. The nanoparticle of claim 7, wherein said positron emission imaging agents include a radioisotope that emit positrons with energies of 511 keV selected from the group consisting of: copper-64 (⁶⁴Cu); gallium-68 (⁶⁸Ga); rubidium-82 (⁸²Rb); bromine-77 (⁷⁷Br); zirconium-89 (⁸⁹Zr); arsenic-71 (⁷¹As); arsenic-72 (⁷²As); arsenic-74 (⁷⁴As); yttrium-86 (⁸⁶Y); yttrium-88 (⁸⁸Y); iodine-124 (¹²⁴I); and combinations thereof.

10. The nanoparticle of claim 6, wherein said radiotherapeutic agent is selected from the group consisting of: radium-223 (²²³Ra); yttrium-90 (⁹⁰Y); lutetium-177 (¹⁷⁷Lu); iodine-131 (¹³¹I); astatine-211 (²¹¹At); bismuth-212 (²¹²Bi); bismuth-213 (²¹³Bi); lead-212 (²¹²Pb); actinium-225 (²²⁵Ac); holmium-166 (¹⁶⁶Ho); samarium-153 (¹⁵³Sm); phosphorus-32 (³²P); phosphorus-33 (³³P); and combinations thereof.

11. The nanoparticle of claim 6, wherein said preselected agent includes both an imaging agent and a radiotherapeutic agent.

12. The nanoparticle of claim 11, wherein said imaging agent is selected from the group consisting of: copper-67 (⁶⁷Cu); lutetium-177 (¹⁷⁷Lu); rhenium-186 (¹⁸⁶Rh); rhenium-188 (¹⁸⁸Rh); technetium-99m (⁹⁹mTc); indium-111 (¹¹¹In); gadolinium-153 (¹⁵³ Gd); copper-64 (⁶⁴Cu); gallium-68 (⁶³Ga); rubidium-82 (⁸²Rb); bromine-77 (⁷⁷Br); zirconium-89 (⁸⁹Zr); arsenic-71 (⁷¹As); arsenic-72 (⁷²As); arsenic-74 (⁷⁴ As); yttrium-86 (⁸⁶Y); yttrium-88 (⁸⁸Y); iodine-124 (¹²⁴I); and combinations thereof; and said therapeutic agent is a radiotherapeutic agent selected from the group consisting of: radium-223 (²²³ Ra); yttrium-90 (⁹⁰Y); lutetium-177 (¹⁷⁷Lu); iodine-131 (¹³¹I); astatine-211 (²¹¹At); bismuth-212 (²¹²Bi); bismuth-213 (²¹³Bi); lead-212 (²¹²Pb); actinium-225 (²²⁵Ac); holmium-166 (¹⁶⁶Ho); samarium-153 (¹⁵³Sm); phosphorus-32 (³²P); phosphorus-33 (³³P); and combinations thereof.

13. The nanoparticle of claim 6, wherein said preselected agent is a metal phosphate that includes a metal or metal cation selected from the group consisting of: Group IA metals, Group IIA metals, Group III-A metals, Group I-B metals, Group II-B metals, Group III-B metals, Group IV-B metals, Group V-B metals, Group VI-B metals, Group VII-B metals, and combinations thereof.

14. The nanoparticle of claim 6, wherein said fluorescence agent includes fluorescein or fluorescein isocyanate.

15. The nanoparticle of claim 6, wherein said redox agent includes hexacyanoferrate (II) or hexacyanoferrate (III).

16. A method for making a functionalized apoferritin nanoparticle, characterized by the step of:
surrounding a preselected agent having a first preselected functionality with an apoferritin nanoparticle having a functionalized outer surface, said functionalized outer surface including at least one preselected surface member.

17. The method of claim 16, wherein the step of surrounding said preselected agent includes disassembling said functionalized apoferritin nanoparticle and reassembling same to surround a quantity of said preselected agent.

18. The method of claim 16, wherein the step of surrounding said preselected agent includes diffusing a preselected quantity of said preselected agent into said apoferritin nanoparticle.

19. The method of claim 16, further comprising releasing a quantity of at least one metal or metal cation from said functionalized apoferritin nanoparticle to generate an electrochemical signal for measurement of same.

20. The method of claim 16, wherein said preselected surface member is attached to said functionalized outer surface using a biotinylation process.

21. A biosensor, comprising:
an apoferritin nanoparticle that includes a functionalized outer surface, surrounding a preselected agent.

22. The biosensor of claim 21, wherein said preselected agent includes a member selected from the group consisting of: metal containing agent; imaging agent; magnetic agent; contrast agent; electrochemical agent; colorimetric agent; optically active agent; therapeutic agent; redox agent; and combinations thereof.

23. The biosensor of claim 21, further including an electrode configured with a transducer, said electrode is operatively coupled to said nanoparticle for detecting said preselected agent in a preselected detection event.

24. The biosensor of claim 23, wherein said biosensor is an immunoassay biosensor and said preselected detection event includes an antibody-antigen binding event.

25. The biosensor of claim 23, wherein said detection event is a nucleic acid binding event for detection of nucleic acid in an immunoassay.

26. The biosensor of claim 23, wherein said detection event is a protein binding event for detection of protein in a protein assay.

27. The biosensor of claim 23, wherein said biosensor includes a strip member that includes an immobilized antibody, said immobilized antibody configured to selectively bind with a preselected target antigen when contacted thereby;
said antigen configured to further complex with a preselected antibody attached to said functionalized outer surface of said nanoparticle in an immunoassay detection event;
whereby said antigen is quantified in conjunction with said preselected agent.
